(19) United States
(12) Patent Application Publication
(10) Pub. No.: US 2022/0235030 A1
(43) Pub. Date: Jul. 28, 2022

(54) SELECTIVE HISTAMINE H3 ANTAGONIST ACID ADDITION SALTS AND PROCESS FOR THE PREPARATION THEREOF

(71) Applicant: RICHTER GEDEON NYRT., Budapest (HU)

(72) Inventors: Ferenc SEBÕK, Gomba (HU); Anikó MEISZTERICS, Budapest (HU); Ádám DEMETER, Budapest (HU)

(21) Appl. No.: 17/615,176

(22) PCT Filed: May 29, 2020
(86) PCT No.: PCT/IB2020/055105

§ 371 Date: Nov. 30, 2021

(30) Foreign Application Priority Data
May 31, 2019 (HU) P1900193

(51) Int. Cl. C07D 401/12

(52) U.S. Cl. CPC C07D 401/12; C07B 2200/13

(57) ABSTRACT

The present invention relates to physically and chemically stable salts of the selective histamine preceptor antagonist compound of 1-[4-(4-{3-[(2R)-2-methyl-pyrrolidin-1-yl]-propoxy}-phenoxy)-piperidin-1-yl]-ethanone of formula (1) and/or polymorphs thereof and/or hydrates/solvates thereof, the process for the preparation thereof, pharmaceutical compositions comprising them, and for use in the treatment and/or prevention of conditions requiring the modulation of histamine H₃ receptors (e.g. Alzheimer's disease, obesity, schizophrenia, miochardial ischaemia, migraine, autism spectrum disorder).

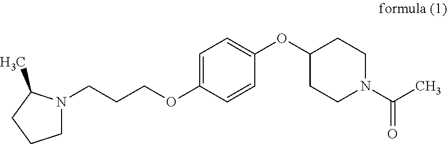 [SMILES: CC(=O)N1CCC(Oc2ccc(OCCCN3CCC[C@H]3C)cc2)CC1]

### THE FIELD OF THE INVENTION

The present invention relates to physically and chemically stable salts of the selective histamine H₃ receptor antagonist compound of 1-[4-(4-{3-[(2R)-2-methyl-pyrrolidin-1-yl]-propoxy}-phenoxy)-piperidin-1-yl]-ethanone of formula (1)

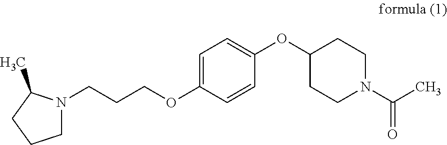 [SMILES: CC(=O)N1CCC(Oc2ccc(OCCCN3CCC[C@H]3C)cc2)CC1]

and/or polymorphs thereof and/or hydrates/solvates thereof, the process for the preparation thereof, pharmaceutical compositions comprising them, and for use in the treatment and/or prevention of conditions requiring the modulation of histamine H₃ receptors (e.g. Alzheimer's disease, obesity, schizophrenia, myocardial ischaemia, migraine, autism spectrum disorder).

### THE BACKGROUND OF THE INVENTION

The histamine H₃ receptor antagonists were extensively studied aiming to produce drugs that would enable the treatment of different diseases, such as Alzheimer's disease, obesity, schizophrenia, myocardial ischaemia, migraine, nasal congestion etc. (Leurs et al., Nat. Rev. Drug. Disc. 2005, 4(2):107-120; Berlin et al., J. Med. Chem. 2011, 54(1):26-53). Numerous compound showed promising preclinical results and entered clinical phase in diseases such as excessive daytime sleepiness (EDS) associated with Parkinson's disease, obstructive sleep apnea, epilepsy, schizophrenia, dementia, and attention deficit hyperactivity disorder (Kuhne et al., Exp. Opin. Inv. Drugs 2011, 20(12):1629-1648). It has been suggested that histamine H₃ receptor antagonists/inverse agonists may also be suitable for pharmacotherapeutic treatment of sleep disorders (Barbier and Bradbury, CNS Neurol. Disord. Drug Targets 2007, 6(1):31-43), but so far, only one histamine H₃ receptor antagonist, pitolisant (under the Wakix brand), has been granted marketing authorization for the treatment of narcolepsy with or without cataplexy in adults (Kollb-Sielecka et al., Sleep Med. 2017, 33:125-129).

WO 2014/136075 describes the synthesis of chemically modifiable, selective and drug-like H₃ antagonists and inverse agonists. The preparation and characterization of such phenoxypiperidine-derived compounds are disclosed therein that bind to H₃ receptor with high affinity and high selectivity and are drug-like.

Among the compounds disclosed in WO 2014/136075, the hydrochloride salt of 1-[4-(4-{3-[(2R)-2-methyl-pyrrolidin-1-yl]-propoxy}-phenoxy)-piperidin-1-yl]-ethanone of formula (1) is highlighted. In the preparation of the compound as described in Example 11, the starting material was 4-(4-{3-[(2R)-2-methyl-pyrrolidin-1-yl]-propoxy}-phenoxy)-piperidine dihydrochloride salt. After the base is released, the reaction mixture is treated with acetyl chloride in dichloromethane, and after the aqueous extraction work-up of the reaction mixture, the dried solution of the resulting base of formula (1) in dichloromethane was evaporated. To a solution of the crude product in dichloromethane excess hydrochloric acid in ethyl acetate was added. The precipitate was filtered off with ethyl acetate and washed with diethyl ether to give a crystalline product, the hydrochloride salt of 1-[4-(4-{3-[(2R)-2-methyl-pyrrolidin-1-yl]-propoxy}-phenoxy)-piperidin-1-yl]-ethanone.

A general requirement for active ingredients in the development of a pharmaceutical composition is that the active ingredient has the appropriate physical, physico-chemical and chemical parameters. Examples of such parameters include solubility, in particular water solubility. Another important feature that should be taken into account in industrial-scale production is the easy handling and the good isolability, which is extremely important for the economicalness of the manufacturing process. A further important aspect is that the solid form of the active ingredient has appropriate physical and chemical stability, for example, not hygroscopic, and does not degrade significantly. Furthermore, different polymorphic forms of a given salt may have different solid phase characteristics, physical and chemical stability.

From a drug development perspective, the water-binding tendency of a substance, the degree of hygroscopicity (ability of absorbency), is of paramount importance, since ambient humidity means a meaningful interaction in addition to the temperature. The degree of hygroscopicity of active ingredients affects the handling, storage, stability, formulability and many other qualities of the substance. There are several approaches and methods to characterize the hygroscopic properties of the active ingredients, and to categorize the degree of hygroscopicity, which is summarized in detail by Newman et al. (Newman et al., J. Pharm. Sci. 2007, 97(3):1047-1059). Typically, non-hygroscopic, slightly hygroscopic, moderately hygroscopic, very hygroscopic, as well as deliquescent categories are used in the literature, while in the pharmacopeia (European Pharmacopeia 9.0, 5.11 Character Section in Monographs) the less hygroscopic, hygroscopic, highly hygroscopic and deliquescent categories are used depending on the weight gain at the given temperature and relative humidity under the test conditions, in a given time. There are static and dynamic measurement methods for the investigation of hygroscopic tendency. Among the dynamic measurements Dynamic Vapor Sorption (DVS) analysis is a technique commonly used in the pharmaceutical industry, which typically measures mass change of the substance (sorption and desorption curve) as a function of relative humidity in isothermic conditions, from which the nature, mechanism and phase transitions of the sorption process can be inferred.

For testing hygroscopicity of active substances it is particularly important to determine whether the substance is susceptible to deliquescence, i.e. what is the point at which the solid material is in dissolved state when interacting with the ambient humidity (Mauer et al., Pharm. Dev. Techn. 2010, 15(6):582-594). Deliquescence of the substance occurs when the relative humidity (RH) reaches or exceeds the critical relative humidity (CRH) when a film corresponding to a saturated solution of the substance is formed on the surface of the solid substance. By further increasing the humidity the substance continuously takes up moisture, leading to drastic weight gain due to the complete dissolution of the material and dilution of the resulting solution. Even a slight surface deliquescence of the substance might have a significant effect on the chemical stability of the compound, since typically in case of compounds with acidic or basic characteristic such microenvironment might occur that leads to the degradation of acid or alkali-sensitive compounds. Deliquescence and strong ability to absorb moisture of the crystalline drugs are typically due to their good solubility.

Determination of the critical relative humidity is feasible by gravimetric method, e.g. with DVS, where relative humidity is changed in suitably selected steps and a sufficiently long time is used to the onset of quasi-equilibrium. After reaching the critical relative humidity, the sorption curve shows a more or less sharp change in the slope, typically followed by a monotonous rise and a significant increase in mass, the extent of which and the shape of the sorption curve cannot be associated with the formation of a hydrate form.

### SUMMARY OF THE INVENTION

The base form of the salts of the present invention, the 1-[4-(4-{3-[(2R)-2-methyl-pyrrolidin-1-yl]-propoxy}-phenoxy)-piperidin-1-yl]-ethanone of formula (1), cannot be isolated in crystalline form, but as oil.

The aim was to obtain a solid form (salt and/or polymorph) of 1-[4-(4-{3-[(2R)-2-methyl-pyrrolidin-1-yl]-propoxy}-phenoxy)-piperidin-1-yl]-ethanone which possesses appropriate properties with regard to the above mentioned aspects, exhibiting adequate physical and chemical stability, slightly hygroscopic, not deliquescent, thereby its isolation is facilitated, handling is better and has excellent solubility.

It has been found during the preparation of the crystalline form of the hydrochloride acid addition salts of the 1-[4-(4-{3-[(2R)-2-methyl-pyrrolidin-1-yl]-propoxy}-phenoxy)-piperidin-1-yl]-ethanone base, that two crystalline polymorphs (Form A and Form B) of the monohydrochloride stoichiometry can be produced. In addition, the crystalline dihydrochloride salt of the compound can also be produced besides the monohydrochloride.

Surprisingly, it has been found that in contrast to the 1-[4-(4-{3-[(2R)-2-methyl-pyrrolidin-1-yl]-propoxy}-phenoxy)-piperidin-1-yl]-ethanone monohydrochloride and dihydrochloride salts the novel dihydrobromide, sulfate, oxalate, monocitrate and dicitrate salts have outstanding properties, are less hygroscopic, easier to be isolated, their physical and chemical stability are more favorable, and have excellent solubility. All of these advantageous properties of the novel 1-[4-(4-{3-[(2R)-2-methyl-pyrrolidin-1-yl]-propoxy}-phenoxy)-piperidin-1-yl]-ethanone dihydrobromide, sulfate, oxalate, monocitrate, and dicitrate salts make them suitable for the development of a pharmaceutical composition for the treatment of diseases targeting the selective modulation of H₃ receptor.

The present invention relates to dihydrobromide, sulfate, oxalate, monocitrate and dicitrate salts of 1-[4-(4-{3-[(2R)-2-methyl-pyrrolidin-1-yl]-propoxy}-phenoxy)-piperidin-1-yl]-ethanone, and/or polymorphs thereof and/or hydrates/solvates thereof, the process for the preparation thereof, pharmaceutical compositions comprising them, and the use thereof in the treatment and/or prevention of conditions requiring the modulation of histamine H₃ receptors (e.g. Alzheimer's disease, obesity, schizophrenia, myocardial ischaemia, migraine, autism spectrum disorder).

### BRIEF DESCRIPTION OF THE FIGURES

FIG. 1 X-ray powder diffraction (XRPD) pattern of 1-[4-(4-{3-[(2R)-2-methyl-pyrrolidin-1-yl]-propoxy}-phenoxy)-piperidin-1-yl]-ethanone monohydrochloride salt Form A (Example 6).

FIG. 2 Dynamic vapor sorption (DVS) isotherm plot of 1-[4-(4-{3-[(2R)-2-methyl-pyrrolidin-1-yl]-propoxy}-phenoxy)-piperidin-1-yl]-ethanone monohydrochloride salt Form A (Example 6).

FIG. 3 X-ray powder diffraction (XRPD) pattern of 1-[4-(4-{3-[(2R)-2-methyl-pyrrolidin-1-yl]-propoxy}-phenoxy)-piperidin-1-yl]-ethanone monohydrochloride salt Form B (Example 7).

FIG. 4 Infrared spectrum (IR) of 1-[4-(4-{3-[(2R)-2-methyl-pyrrolidin-1-yl]-propoxy}-phenoxy)-piperidin-1-yl]-ethanone monohydrochloride salt Form B (Example 7).

FIG. 5 Raman spectrum (Raman) of 1-[4-(4-{3-[(2R)-2-methyl-pyrrolidin-1-yl]-propoxy}-phenoxy)-piperidin-1-yl]-ethanone monohydrochloride salt Form B (Example 7).

FIG. 6 Dynamic vapor sorption (DVS) isotherm plot of 1-[4-(4-{3-[(2R)-2-methyl-pyrrolidin-1-yl]-propoxy}-phenoxy)-piperidin-1-yl]-ethanone monohydrochloride salt Form B (Example 7).

FIG. 7 X-ray powder diffraction (XRPD) pattern of 1-[4-(4-{3-[(2R)-2-methyl-pyrrolidin-1-yl]-propoxy}-phenoxy)-piperidin-1-yl]-ethanone dihydrochloride salt (Example 2).

FIG. 8 Termogravimetric (TG) curve of 1-[4-(4-{3-[(2R)-2-methyl-pyrrolidin-1-yl]-propoxy}-phenoxy)-piperidin-1-yl]-ethanone dihydrochloride salt (Example 2).

FIG. 9 Differential scanning calorimetry (DSC) thermogram of 1-[4-(4-{3-[(2R)-2-methyl-pyrrolidin-1-yl]-propoxy}-phenoxy)-piperidin-1-yl]-ethanone dihydrochloride salt (Example 2).

FIG. 10 Infrared spectrum (IR) of 1-[4-(4-{3-[(2R)-2-methyl-pyrrolidin-1-yl]-propoxy}-phenoxy)-piperidin-1-yl]-ethanone dihydrochloride salt (Example 2).

FIG. 11 Raman spectrum (Raman) of 1-[4-(4-{3-[(2R)-2-methyl-pyrrolidin-1-yl]-propoxy}-phenoxy)-piperidin-1-yl]-ethanone dihydrochloride salt (Example 2).

FIG. 12 Dynamic vapor sorption (DVS) isotherm plot of 1-[4-(4-{3-[(2R)-2-methyl-pyrrolidin-1-yl]-propoxy}-phenoxy)-piperidin-1-yl]-ethanone dihydrochloride salt (Example 2).

Hiba! A hivatkozási forrás nem található. Dynamic vapor sorption curves of the salts tested (relative weight change %−relative humidity %) at 25° C. (a) deliquescent salts (b) not deliquescent salts.

FIG. 15 X-ray powder diffraction (XRPD) pattern of 1-[4-(4-{3-[(2R)-2-methyl-pyrrolidin-1-yl]-propoxy}-phenoxy)-piperidin-1-yl]-ethanone monocitrate salt Form A (Example 17).

FIG. 16 Infrared spectrum (IR) of 1-[4-(4-{3-[(2R)-2-methyl-pyrrolidin-1-yl]-propoxy}-phenoxy)-piperidin-1-yl]-ethanone monocitrate salt Form A (Example 17).

FIG. 17 Raman spectrum (Raman) of 1-[4-(4-{3-[(2R)-2-methyl-pyrrolidin-1-yl]-propoxy}-phenoxy)-piperidin-1-yl]-ethanone monocitrate salt Form A (Example 17).

FIG. 18 X-ray powder diffraction (XRPD) pattern of 1-[4-(4-{3-[(2R)-2-methyl-pyrrolidin-1-yl]-propoxy}-phenoxy)-piperidin-1-yl]-ethanone monocitrate salt Form B (Example 18).

FIG. 19 Infrared spectrum (IR) of 1-[4-(4-{3-[(2R)-2-methyl-pyrrolidin-1-yl]-propoxy}-phenoxy)-piperidin-1-yl]-ethanone monocitrate salt Form B (Example 18).

FIG. 20 Raman spectrum (Raman) of 1-[4-(4-{3-[(2R)-2-methyl-pyrrolidin-1-yl]-propoxy}-phenoxy)-piperidin-1-yl]-ethanone monocitrate salt Form B (Example 18).

FIG. 21 Dynamic vapor sorption (DVS) isotherm plot of 1-[4-(4-{3-[(2R)-2-methyl-pyrrolidin-1-yl]-propoxy}-phenoxy)-piperidin-1-yl]-ethanone monocitrate salt (Example 17).

FIG. 22 X-ray powder diffraction (XRPD) pattern of 1-[4-(4-{3-[(2R)-2-methyl-pyrrolidin-1-yl]-propoxy}-phenoxy)-piperidin-1-yl]-ethanone dicitrate salt (Example 20).

FIG. 23 Infrared spectrum (IR) of 1-[4-(4-{3-[(2R)-2-methyl-pyrrolidin-1-yl]-propoxy}-phenoxy)-piperidin-1-yl]-ethanone dicitrate salt (Example 20).

FIG. 24 Raman spectrum (Raman) of 1-[4-(4-{3-[(2R)-2-methyl-pyrrolidin-1-yl]-propoxy}-phenoxy)-piperidin-1-yl]-ethanone dicitrate salt (Example 20).

FIG. 25 Dynamic vapor sorption (DVS) isotherm plot of 1-[4-(4-{3-[(2R)-2-methyl-pyrrolidin-1-yl]-propoxy}-phenoxy)-piperidin-1-yl]-ethanone dicitrate salt (Example 20).

FIG. 26 X-ray powder diffraction (XRPD) pattern of 1-[4-(4-{3-[(2R)-2-methyl-pyrrolidin-1-yl]-propoxy}-phenoxy)-piperidin-1-yl]-ethanone dihydrobromide salt (Example 9).

FIG. 27 Infrared spectrum (IR) of 1-[4-(4-{3-[(2R)-2-methyl-pyrrolidin-1-yl]-propoxy}-phenoxy)-piperidin-1-yl]-ethanone dihydrobromide salt (Example 9).

FIG. 28 Raman spectrum (Raman) of 1-[4-(4-{3-[(2R)-2-methyl-pyrrolidin-1-yl]-propoxy}-phenoxy)-piperidin-1-yl]-ethanone dihydrobromide salt (Example 9).

FIG. 29 Dynamic vapor sorption (DVS) isotherm plot of 1-[4-(4-{3-[(2R)-2-methyl-pyrrolidin-1-yl]-propoxy}-phenoxy)-piperidin-1-yl]-ethanone dihydrobromide salt (Example 9).

FIG. 30 X-ray powder diffraction (XRPD) pattern of 1-[4-(4-{3-[(2R)-2-methyl-pyrrolidin-1-yl]-propoxy}-phenoxy)-piperidin-1-yl]-ethanone sulfate salt (Example 10).

FIG. 31 Infrared spectrum (IR) of 1-[4-(4-{3-[(2R)-2-methyl-pyrrolidin-1-yl]-propoxy}-phenoxy)-piperidin-1-yl]-ethanone sulfate salt (Example 10).

FIG. 32 Raman spectrum (Raman) of 1-[4-(4-{3-[(2R)-2-methyl-pyrrolidin-1-yl]-propoxy}-phenoxy)-piperidin-1-yl]-ethanone sulfate salt (Example 10).

FIG. 33 Dynamic vapor sorption (DVS) isotherm plot of 1-[4-(4-{3-[(2R)-2-methyl-pyrrolidin-1-yl]-propoxy}-phenoxy)-piperidin-1-yl]-ethanone sulfate salt (Example 10).

FIG. 34 X-ray powder diffraction (XRPD) pattern of 1-[4-(4-{3-[(2R)-2-methyl-pyrrolidin-1-yl]-propoxy}-phenoxy)-piperidin-1-yl]-ethanone oxalate salt (Example 13).

FIG. 35 Infrared spectrum (IR) of 1-[4-(4-{3-[(2R)-2-methyl-pyrrolidin-1-yl]-propoxy}-phenoxy)-piperidin-1-yl]-ethanone oxalate salt (Example 13).

FIG. 36 Raman spectrum (Raman) of 1-[4-(4-{3-[(2R)-2-methyl-pyrrolidin-1-yl]-propoxy}-phenoxy)-piperidin-1-yl]-ethanone oxalate salt (Example 13).

FIG. 37 Dynamic vapor sorption (DVS) isotherm plot of 1-[4-(4-{3-[(2R)-2-methyl-pyrrolidin-1-yl]-propoxy}-phenoxy)-piperidin-1-yl]-ethanone oxalate salt (Example 13).

### DETAILED DESCRIPTION OF THE INVENTION

The base form of the salts of the present invention, the 1-[4-(4-{3-[(2R)-2-methyl-pyrrolidin-1-yl]-propoxy}-phenoxy)-piperidin-1-yl]-ethanone of formula (1), cannot be isolated in crystalline form, but as oil. The base according to the procedure described in Example 11 of WO 2014/136075 can be obtained by evaporating the dichloromethane solution of the resulting product or, after isolation of the hydrochloride salt—in a manner obvious to the skilled person—by base releasing.

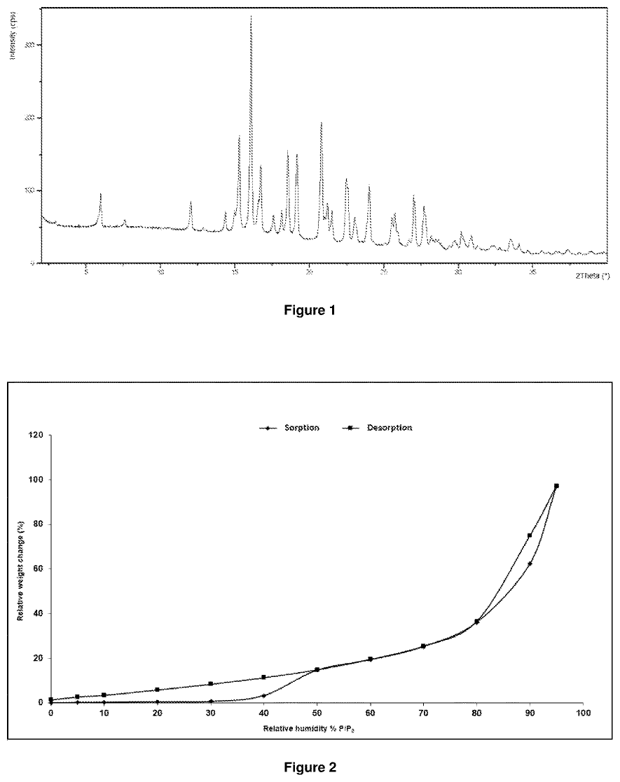

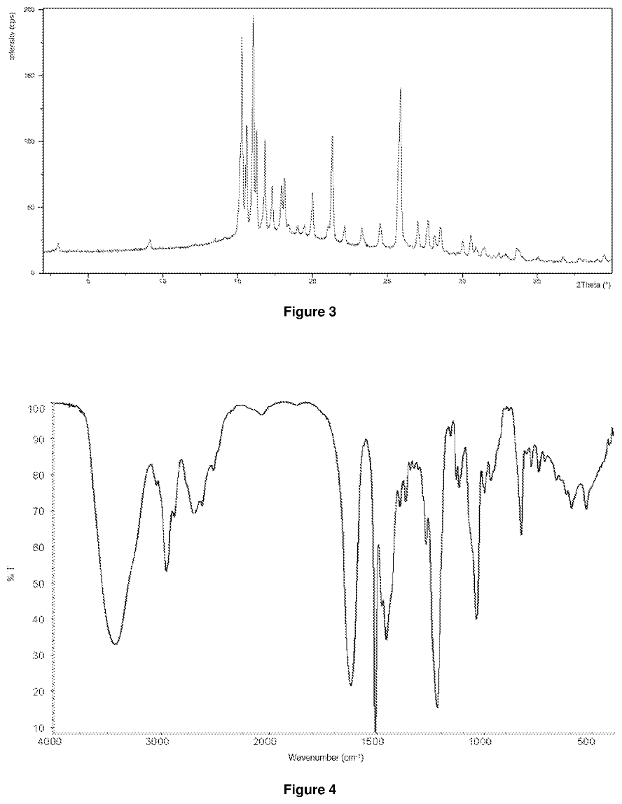

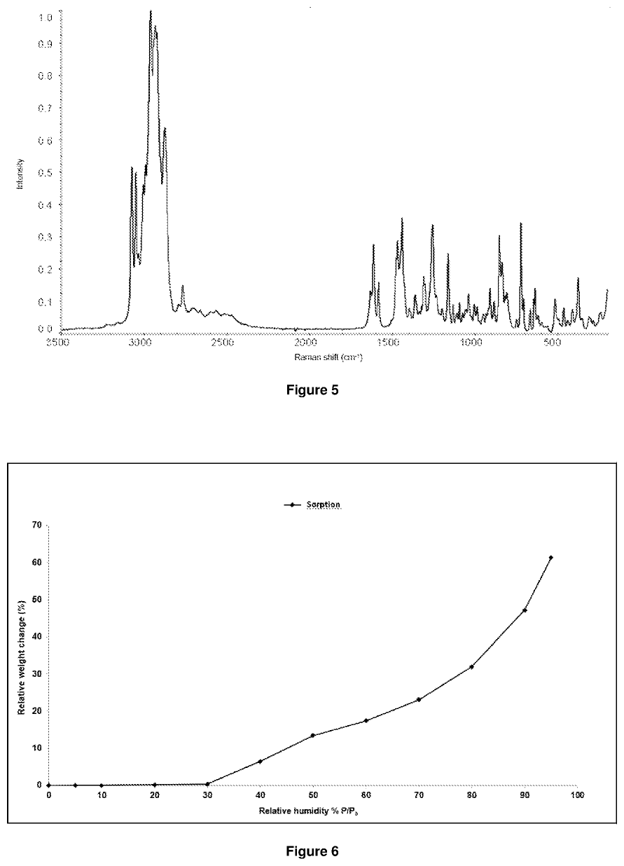

The hydrochloride acid addition salts of 1-[4-(4-{3-[(2R)-2-methyl-pyrrolidin-1-yl]-propoxy}-phenoxy)-piperidin-1-yl]-ethanone base (Example 1) are prepared in crystalline form (Example 2 to Example 8). It has been found that two crystalline polymorphs (Form A and Form B) of the salt characterized by monohydrochloride stoichiometry can be produced (Example 4 to Example 8), of which X-ray powder diffraction (XRPD) patterns, infrared (IR) and Raman spectra, and dynamic vapor sorption (DVS) isotherm plot are shown in FIG. 1 to FIG. 6. Both monohydrochloride polymorphs (Form A and Form B) are highly hygroscopic and prone to deliquescence. Based on the DVS analysis at 25° C., Form A has, above 40% relative humidity, and Form B has, yet above 30% relative humidity, a high, continuous weight gain in the sorption process which is caused by the deliquescence of the substance.

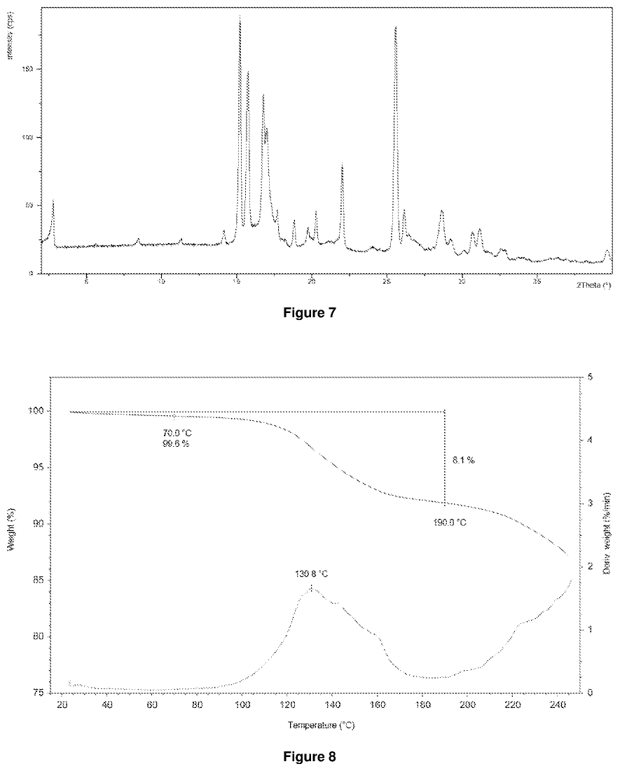

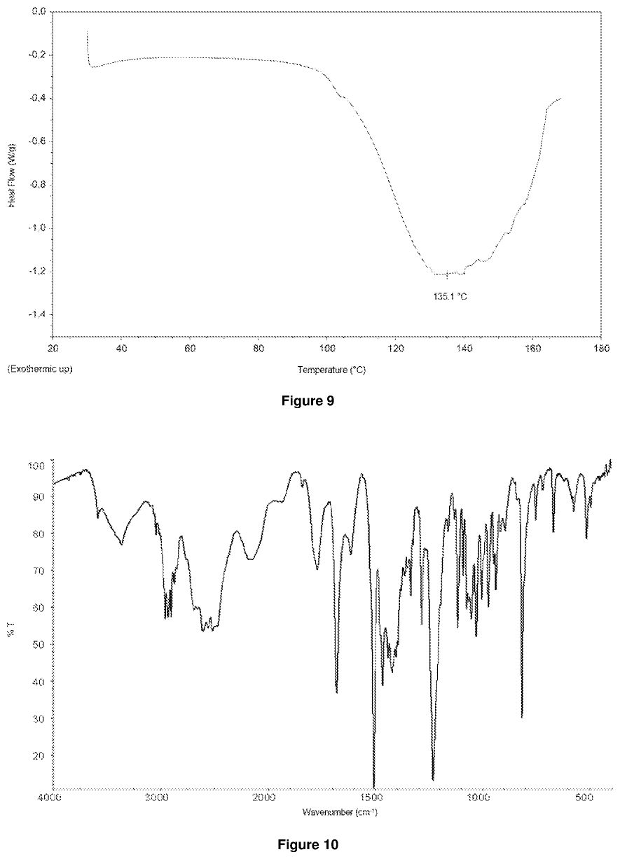

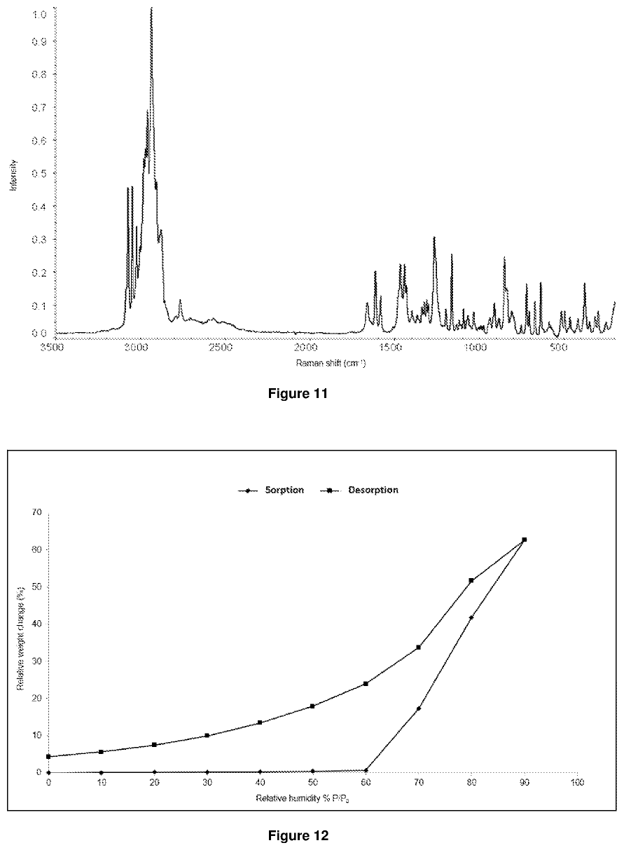

In further experiments, it was found that crystalline dihydrochloride salt (diHCl) of the compound can also be produced (Example 2 and Example 3) in addition to the monohydrochloride, of which X-ray powder diffraction (XRPD) pattern, termogravimetric (TG) curve, differential scanning calorimetry (DSC) thermogram, infrared (IR) and Raman spectra, and dynamic vapor sorption (DVS) isotherm plot are shown in FIG. 7 to FIG. 12. The compound of 1-[4-(4-{3-[(2R)-2-methyl-pyrrolidin-1-yl]-propoxy}-phenoxy)-piperidin-1-yl]-ethanone contains a single strongly basic center (pyrrolidine nitrogen), which is capable of forming stoichiometric salt with equimolar hydrochloride, thus the formation of dihydrochloride stoichiometry is not expected in view of the acid/base character of the compound. Based on TG and DSC analysis, the second molar amount of hydrochloride is less strongly bound to the crystal lattice, behaving as a volatile component. The compound is thermally poorly stable, according to the TG analysis the loss of volatile HCl can already be observed at room temperature, but becomes intensive at about 70 to 80° C. (FIG. 8). Parallelly to this process, according to the DSC and microscopic analysis, the sample starting from approx. 100° C. melts during decomposition (FIG. 9).

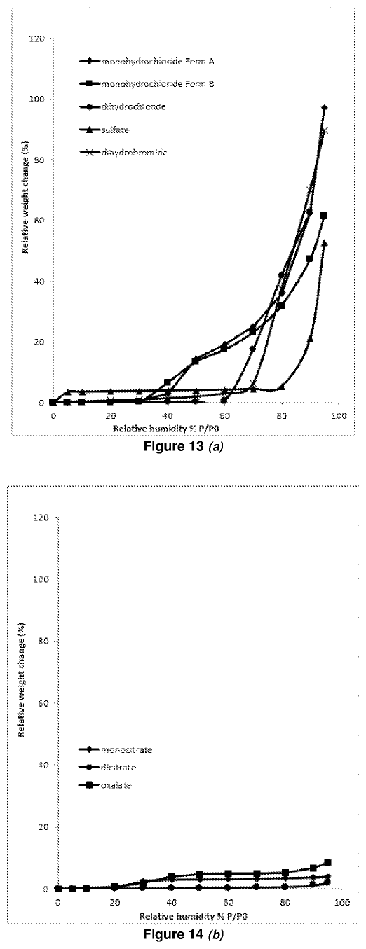

A further disadvantage of the dihydrochloride form is that, it is highly hygroscopic, according to the DVS (dynamic vapor sorption) analysis at 25° C., a significant monotonic weight increase is observed on the sorption curve above 60% relative humidity, showing the deliquescence of the substance (FIG. 12).

The hygroscopic nature of the mono- and dihydrochloride salts of 1-[4-(4-{3-[(2R)-2-methyl-pyrrolidin-1-yl]-propoxy}-phenoxy)-piperidin-1-yl]-ethanone poses many issues in terms of the pharmaceutical development, handling, storing, stability and formulability of the compound. It has been observed that hydrochloride salts are already susceptible to deliquescence under the conditions of isolation, their filtering and handling are thus problematic. The degradation tendency of the substance is also clearly related to its hygroscopic nature, as the deacetylation of the compound may occur due to exposure to acid in the presence of moisture.

It is therefore necessary to produce salts of 1-[4-(4-{3-[(2R)-2-methyl-pyrrolidin-1-yl]-propoxy}-phenoxy)-piperidin-1-yl]-ethanone that are less hygroscopic, easier to handle, physically and chemically more stable than mono- and dihydrochloride salts.

In our experiments, dihydrobromide salt (Example 9), sulfate salt (Example 10 to Example 12), oxalate salt (Example 13 and Example 14), monocitrate salt (Example 15 to Example 18) and dicitrate salt (Example 19 to Example 22) of 1-[4-(4-{3-[(2R)-2-methyl-pyrrolidin-1-yl]-propoxy}-phenoxy)-piperidin-1-yl]-ethanone was prepared in crystalline form, which are more preferred than the mono- and dihydrochloride salts, as these are less hygroscopic (Table 1), thus easier to isolate and handle, and their stability is much more favorable (Table 2). X-ray powder diffraction, IR and Raman data suitable to characterize polymorphs of crystalline salts of 1-[4-(4-{3-[(2R)-2-methyl-pyrrolidin-1-yl]-propoxy}-phenoxy)-piperidin-1-yl]-ethanone are shown in Table 3 to Table 5.

Thus, the present invention relates to pharmaceutically acceptable, less hygroscopic, acid addition salts of 1-[4-(4-{3-[(2R)-2-methyl-pyrrolidin-1-yl]-propoxy}-phenoxy)-piperidin-1-yl]-ethanone that can be formed with organic or inorganic acids and/or polymorphs thereof and/or hydrates/solvates thereof. Examples of acid addition salts that can be formed with such organic or inorganic acids include salts derived from hydrogen bromide, sulfuric acid, oxalic acid, or citric acid.

Preferably, the present invention relates to 1-[4-(4-{3-[(2R)-2-methyl-pyrrolidin-1-yl]-propoxy}-phenoxy)-piperidin-1-yl]-ethanone dihydrobromide, sulfate, oxalate, monocitrate and dicitrate salts and/or polymorphs thereof and/or hydrates/solvates thereof.

The present invention also relates to the preparation of pharmaceutically acceptable, less hygroscopic, acid addition salts of 1-[4-(4-{3-[(2R)-2-methyl-pyrrolidin-1-yl]-propoxy}-phenoxy)-piperidin-1-yl]-ethanone that can be formed with organic or inorganic acids, preferably dihydrobromide, sulfate, oxalate, monocitrate and dicitrate salts thereof, and/or polymorphs thereof and/or hydrates/solvates thereof.

The present invention also relates to 1-[4-(4-{3-[(2R)-2-methyl-pyrrolidin-1-yl]-propoxy}-phenoxy)-piperidin-1-yl]-ethanone dihydrobromide, sulfate, oxalate, monocitrate and dicitrate salts for use in the treatment and/or prevention of conditions requiring the modulation of histamine H₃ receptors.

The present invention relates to the use of 1-[4-(4-{3-[(2R)-2-methyl-pyrrolidin-1-yl]-propoxy}-phenoxy)-piperidin-1-yl]-ethanone salts in the manufacture of a pharmaceutical composition.

The present invention also relates to a pharmaceutical composition comprising a therapeutically effective amount of 1-[4-(4-{3-[(2R)-2-methyl-pyrrolidin-1-yl]-propoxy}-phenoxy)-piperidin-1-yl]-ethanone dihydrobromide, sulfate, oxalate, monocitrate and dicitrate salts together with pharmaceutically acceptable excipients.

The present invention also relates to the use of the pharmaceutical composition of the previous paragraph in the treatment and/or prevention of conditions requiring the modulation of histamine H₃ receptors, preferably in the treatment and/or prevention of autism spectrum disorder.

For example, the preparation of salts from the base can be carried out as follows: the 1-[4-(4-{3-[(2R)-2-methyl-pyrrolidin-1-yl]-propoxy}-phenoxy)-piperidin-1-yl]-ethanone base is dissolved in a suitable solvent or mixture of solvents, followed by the addition of the acid or a salt thereof—formed by a base weaker than 1-[4-(4-{3-[(2R)-2-methyl-pyrrolidin-1-yl]-propoxy}-phenoxy)-piperidin-1-yl]-ethanone—or a solution thereof, to the mixture. In addition, the 1-[4-(4-{3-[(2R)-2-methyl-pyrrolidin-1-yl]-propoxy}-phenoxy)-piperidin-1-yl]-ethanone base can be prepared from a salt thereof, and after releasing the base, after the appropriate separation and/or solvent exchange, the desired salt is formed by addition of the acid, without isolation of the base. If necessary, the reaction mixture is concentrated, the precipitated product is isolated by filtration at room temperature or after cooling, then dried, if necessary, at an appropriate temperature. If necessary, the resulting salt is crystallized by addition of a suitable antisolvent from its solution at room temperature or after reflux, and the precipitated product is isolated by filtration, then dried, if necessary, at an appropriate temperature.
The salts of the present invention can be well isolated and as a result of the process obtainable in high purity, which makes them particularly valuable for pharmaceutical use. In terms of implementation of the present invention, the monocitrate and dicitrate salts are particularly preferred for the preparation of a pharmaceutical composition, in which case the best quality and most stable product is obtained in excellent yields. Monocitrate and dicitrate salts are poorly hygroscopic, do not show deliquescence, their physical and chemical stability, as well as solubility are excellent.
Both citrate salts have a higher melting point than the dihydrochloride salt. It the case of monocitrate, approx. a 15° C., while in the case of dicitrate, approx. a 30° C. of melting point increase can be observed which indicates greater stability and is more advantageous for the preparation of a pharmaceutical composition. The monocitrate salt is stable under normal laboratory conditions in the form of monohydrate (monocitrate Form A), but by increasing the temperature from room temperature to approx. 70 to 90° C. it loses weakly bound structural water and converts to anhydrate form (monocitrate Form B). The dried sample also takes up its stoichiometric water content relatively quickly when interacting with ambient humidity. The dicitrate salt is stable in the form of anhydrate, does not convert to hydrate form, and has in a development view a favorable, sufficiently high melting point.

Comparison of the dynamic vapor sorption curves measured at 25° C. of the investigated salts of 1-[4-(4-{3-[(2R)-2-methyl-pyrrolidin-1-yl]-propoxy}-phenoxy)-piperidin-1-yl]-ethanone is depicted on Hiba! A hivatkozási forrás nem található. that shows the relative weight change (˜percentage change in weight relative to a weight at 0% relative humidity) as a function of relative humidity (RH %).

On the sorption curve of monohydrochloride salt Form A (FIG. 2), about 3% of water is bound up to 40% RH and then liquefies at higher humidity (95% RH—relative weight change: 97%).

On the sorption curve of monohydrochloride salt Form B (FIG. 6), only 0.3% of water is bound up to 30% RH, presumably by surface adsorption, and then liquefies at higher humidity (95% RH—relative weight change: 61%).

On the sorption curve of the dihydrochloride salt (FIG. 12), about 0.7% of water is bound up to 60% RH relative to the dried mass, and then liquefies at higher humidity (at 70% RH already shows 17% relative weight gain, at 90% RH the relative weight change is 63%). In the case of the DVS measurement of the dihydrochloride salt, unlike the general method description, no measurements were made in the measurement cycle at 5% and 95% relative humidity, but this difference does not significantly affect the determination of the onset of deliquescence (see below).

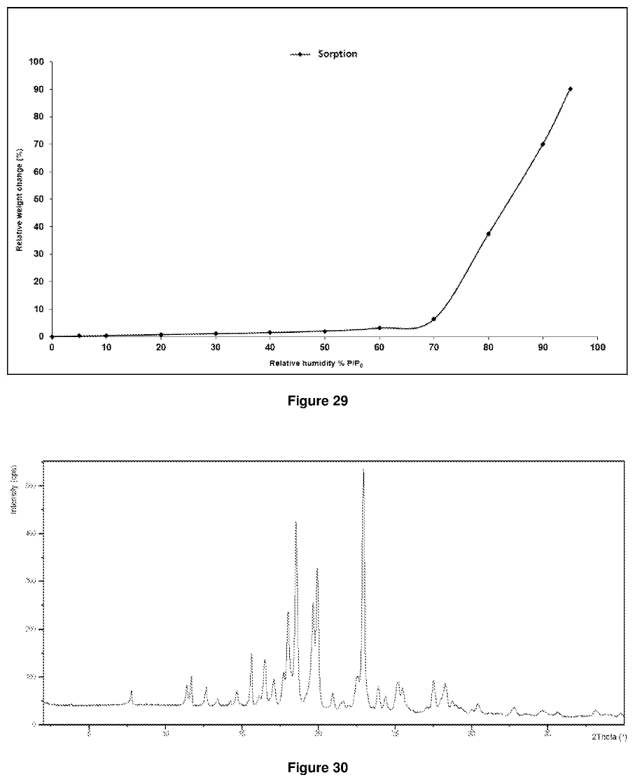

On the sorption curve of the dihydrobromide salt (FIG. 29), it absorbs about 6% moisture up to 70% RH and then liquefies at higher humidity (95% RH— relative weight change: 90%).

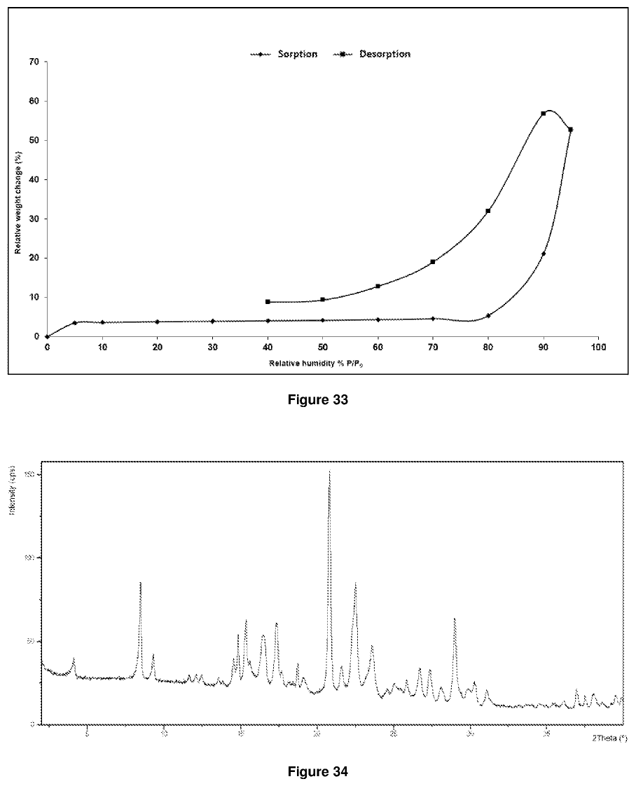

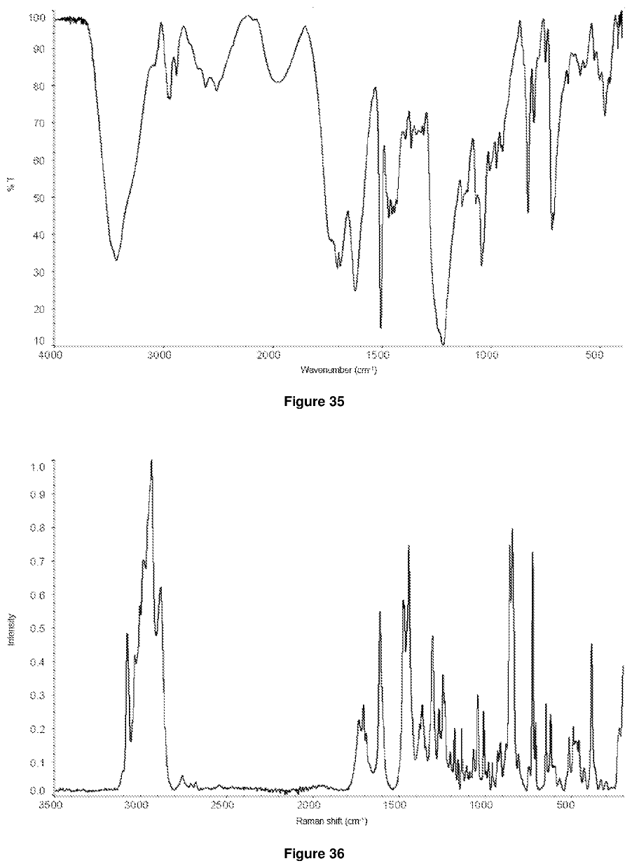

On the sorption curve of the sulfate salt (FIG. 33), about 5% of water is bound up to 80% RH and then liquefies at higher humidity (95% RH— relative weight change: 53%).

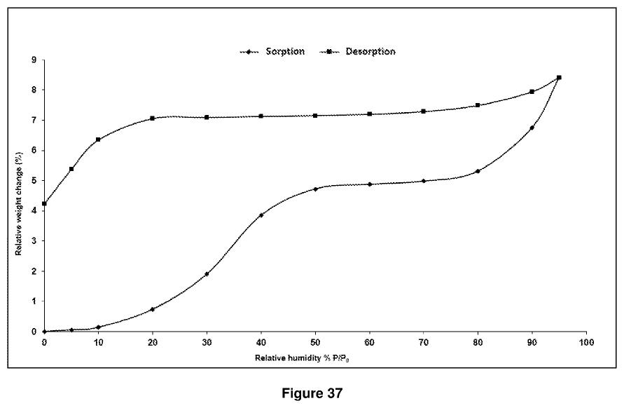

On the sorption curve of the oxalate salt (FIG. 37), a relative weight gain of about 4.7% relative to the dried weight is observed in the 10-50% RH range, which seems to be constant up to approx. 70% RH. This weight change refers to the formation of a hydrate form having a stoichiometry nearly that of a monohydrate. Further water-take up begins above 80% RH, but the oxalate salt does not even show deliquescence above 90% RH under test conditions. In the desorption cycle between 20 to 70% RH, it stabilizes at 7.1 to 7.3% relative weight, indicating the formation of stoichiometry corresponding to the dihydrate form. Thus, the oxalate salt stabilizes in the form of a hydrate having different compositions depending on the humidity.

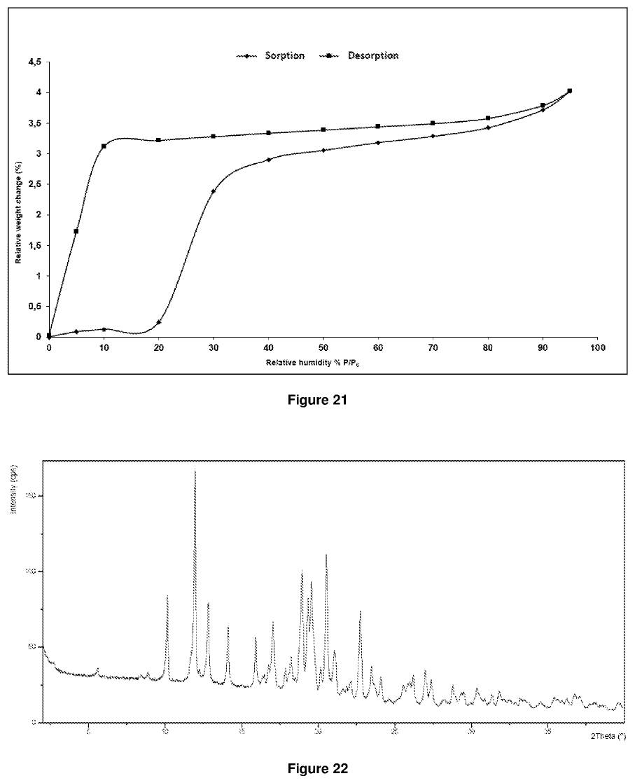

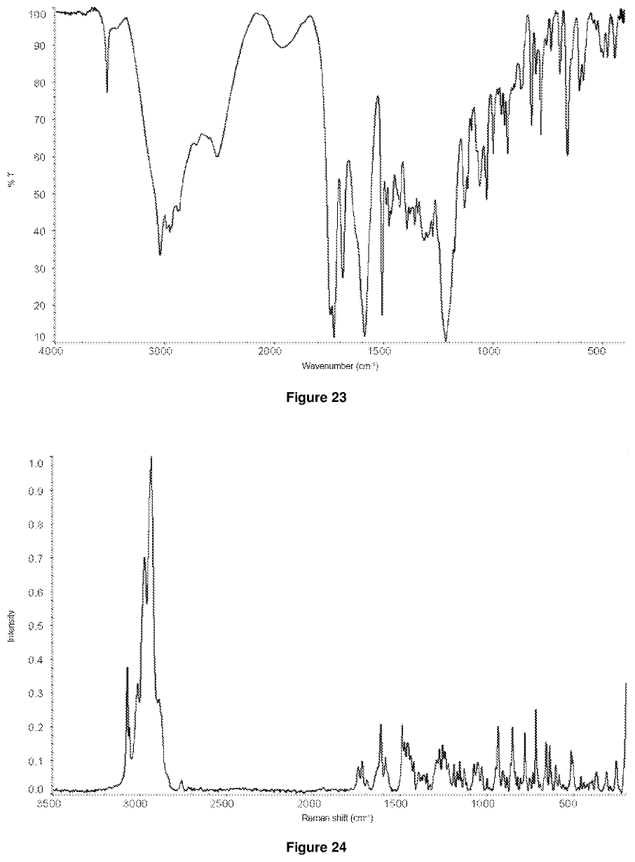

On the DVS curves of the monocitrate salt (FIG. 21) the sample's monohydrate (Form A) and anhydrate (Form B) states can be well isolated. Above 30% RH it takes up 2.6 to 4.3% of water relative to the dried state of the substance, which is close to the theoretical calculated value of monocitrate monohydrate (3.2%). The monohydrate form has proved to be so stable that during desorption the monohydrate Form A is converted to the anhydrate Form B only below 10% RH. The monocitrate salt did not show deliquescence even above 90% RH.

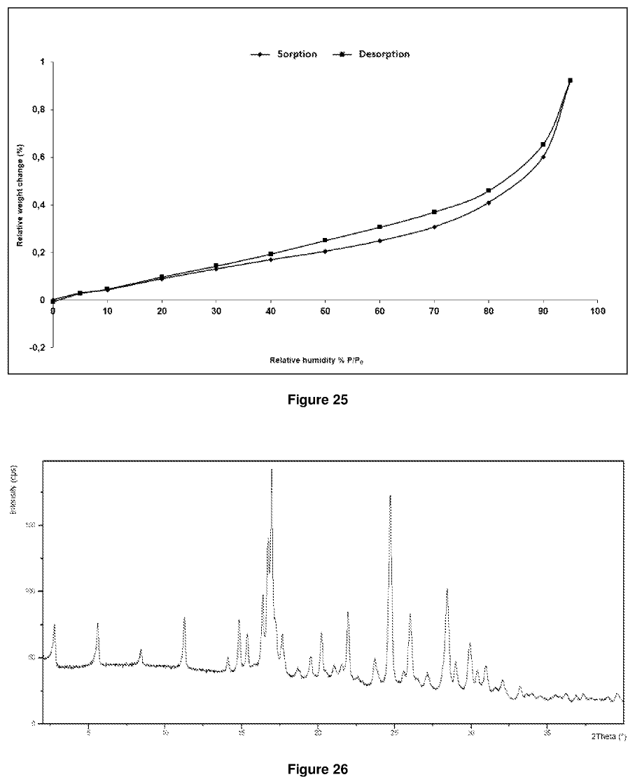

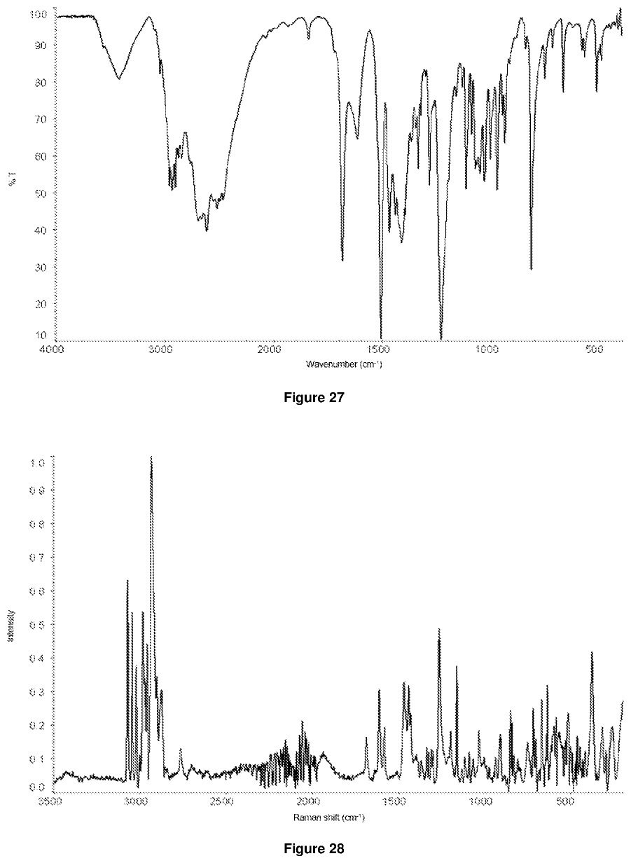

On the sorption curve of the dicitrate salt (FIG. 25), 3.2% of water is bound up to 80% RH, 6.8% up to 90% RH, does not show deliquescence above 90% RH, and its weight reversibly decreases during the desorption phase.

The generally observed hygroscopic nature of the 1-[4-(4-{3-[(2R)-2-methyl-pyrrolidin-1-yl]-propoxy}-phenoxy)-piperidin-1-yl]-ethanone salts is inter alia related to the good solubility thereof. In simulated gastric fluid (SGF without pepsin, pH=1.3), the dihydrochloride salt has a solubility of greater than 59 mM, the solubility of the monocitrate salt is greater than 44 mM, and the solubility of the dicitrate salt is 469 mM.

The deliquescence tendency of each salt is characterized by the critical relative humidity (CRH) value (Table 1), which was determined based on the sorption curves measured according to the measurement parameter settings below, with DVS analysis at 25° C. isotherm conditions, according to the following.

Derivative of the sorption curve was formed on the sorption curve between 10 to 90% RH by determining the differences in relative weight changes relative to 10% RH change:

Δm=m2−m1

where m1 and m2 are the quasi-equilibrium relative mass changes (˜percentage change in weight relative to a weight at 0% relative humidity) for the given percentage of the relative humidity of the sorption curve RH1 and RH2, and

ΔRH=RH2−RH1=10.

If the given sorption step is Δm/ΔRH≥0.5, then RH1 is considered to be the critical relative humidity (CRH) value indicating the end point of the physical stability of the substance. The value thus determined is a good match with the onset of a significant monotonic weight gain observed visually on the sorption curve. Above the critical relative humidity value, it is the process of deliquescence of the substance that determines the weight gain observed on the sorption curve.

TABLE 1
Critical Relative Humidity (CRH) and Δm/ΔRH values based on DVS
analysis at 25° C. characterizing the deliquescence of the tested salts are.

| | Salt | | | | | | | |
|---|---|---|---|---|---|---|---|---|
| | monoHCl | | | | | | mono | |
| | Form B | Form A | diHCl | diHBr | sulfate | oxalate | citrate | dicitrate |
| | CRH | | | | | | | |
| | 30% | 40% | 60% | 70% | 80% | not deliquescent | | |
| | RH₁ | | | | | | | |
| | Δm/ΔRH | | | | | | | |
| 0% | 0.0 | 0.0 | 0.0 | 0.0 | 0.4 | 0.0 | 0.0 | 0.0 |
| 10% | 0.0 | 0.0 | 0.0 | 0.0 | 0.0 | 0.1 | 0.0 | 0.0 |
| 20% | 0.0 | 0.0 | 0.0 | 0.0 | 0.0 | 0.1 | 0.2 | 0.0 |
| 30% | 0.6 | 0.3 | 0.0 | 0.0 | 0.0 | 0.2 | 0.1 | 0.0 |
| 40% | 0.7 | 1.1 | 0.0 | 0.1 | 0.0 | 0.1 | 0.0 | 0.0 |
| 50% | 0.4 | 0.5 | 0.0 | 0.1 | 0.0 | 0.0 | 0.0 | 0.0 |
| 60% | 0.6 | 0.6 | 1.7 | 0.3 | 0.0 | 0.0 | 0.0 | 0.0 |
| 70% | 0.9 | 1.1 | 2.4 | 3.1 | 0.1 | 0.0 | 0.0 | 0.0 |
| 80% | 1.5 | 2.6 | 2.1 | 3.3 | 1.6 | 0.1 | 0.0 | 0.1 |

Compared to monohydrochloride salts, it can be established that diHBr, sulfate salts begin to show deliquescence at significantly higher critical relative humidity, which indicates a reduced hygroscopic tendency associated with their greater physical stability. Surprisingly, the oxalate, monocitrate, and dicitrate salts are not deliquescent under the conditions of the DVS analysis, and are the physically most stable ones.
Increased stability to ambient humidity is beneficial for longer-term physical and chemical stability of the active ingredient. The relationship between the reduced hygroscopic nature and the increased chemical stability associated with it is shown in the most preferred citrate salts in comparison to the dihydrochloride salt.
Table 2 shows the HPLC purity test results of a 10-day solid stress stability study of dihydrochloride, monocitrate and dicitrate salts. It is clear from the results that the dihydrochloride salt is slightly degraded by heat while it degrades significantly under the combined effect of heat and humidity. In contrast, the monocitrate and dicitrate salts are stable under these conditions and are significantly more advantageous.

TABLE 2
HPLC purity test results of a 10-day solid stress stability
study of the dihydrochloride, monocitrate and dicitrate salts

| | Condition | | | | |
|---|---|---|---|---|---|
| | | 40° C., | 50° C., | | |
| | | 75% RH | 75% RH | 50° C. | 75° C. |
| Storage | starting | open | open | sealed | sealed |
| condition | sample | glass | glass | glass | glass |
| Salt form | All contaminant (area %) | | | | |
| dihydrochloride | 0.85% | 59.56% | 51.32% | 0.98% | 1.50% |
| monocitrate | 0.19% | 0.19% | 0.19% | 0.20% | 0.19% |
| dicitrate | 0.23% | 0.24% | 0.25% | 0.23% | 0.24% |

TABLE 3
X-ray powder diffraction characteristics of the 1-[4-(4-
{3-[(2R)-2-methyl-pyrrolidin-1-yl]-propoxy}-phenoxy)-
piperidin-1-yl]-ethanone crystalline salts and polymorphs thereof

| | | |
|---|---|---|
| Peak position | d-spacing | Rel. int. |
| [°2Th.] | [Ä] | [%] |

dihydrochloride

| | | |
|---|---|---|
| 2.8 | 31.5 | 20 |
| 14.2 | 6.3 | 6 |
| 15.2 | 5.8 | 98 |
| 15.8 | 5.6 | 67 |
| 16.8 | 5.3 | 57 |
| 17.0 | 5.2 | 36 |
| 17.7 | 5.0 | 9 |
| 18.8 | 4.7 | 12 |
| 19.7 | 4.5 | 7 |
| 20.3 | 4.4 | 15 |
| 22.0 | 4.0 | 37 |
| 25.6 | 3.5 | 100 |
| 26.1 | 3.4 | 15 |
| 28.7 | 3.1 | 19 |
| 30.7 | 2.9 | 9 |
| 31.2 | 2.9 | 11 |

monohydrochloride Form A

| | | |
|---|---|---|
| 6.0 | 14.7 | 15 |
| 12.1 | 7.3 | 13 |
| 14.4 | 6.2 | 9 |
| 15.0 | 5.9 | 9 |
| 15.3 | 5.8 | 44 |
| 16.1 | 5.5 | 100 |
| 16.7 | 5.3 | 31 |
| 17.6 | 5.0 | 8 |
| 18.1 | 4.9 | 10 |
| 18.6 | 4.8 | 39 |
| 19.2 | 4.6 | 39 |
| 20.8 | 4.3 | 52 |
| 21.2 | 4.2 | 16 |
| 21.5 | 4.1 | 13 |
| 22.5 | 4.0 | 29 |
| 22.6 | 3.9 | 24 |
| 23.0 | 3.9 | 11 |
| 24.0 | 3.7 | 26 |
| 25.5 | 3.5 | 12 |
| 25.7 | 3.5 | 15 |
| 27.0 | 3.3 | 24 |
| 27.7 | 3.2 | 19 |
| 30.2 | 3.0 | 8 |
| 30.9 | 2.9 | 7 |

monohydrochloride Form B

| | | |
|---|---|---|
| 15.3 | 5.8 | 91 |
| 15.6 | 5.7 | 50 |
| 16.0 | 5.5 | 100 |
| 16.3 | 5.5 | 47 |
| 16.8 | 5.3 | 42 |
| 17.3 | 5.1 | 20 |
| 17.9 | 5.0 | 21 |
| 18.1 | 4.9 | 25 |
| 20.0 | 4.4 | 20 |
| 21.3 | 4.2 | 49 |
| 22.1 | 4.0 | 8 |
| 23.3 | 3.8 | 8 |
| 24.5 | 3.6 | 10 |
| 25.9 | 3.4 | 71 |
| 27.0 | 3.3 | 12 |
| 27.7 | 3.2 | 13 |
| 28.1 | 3.2 | 7 |
| 28.5 | 3.1 | 11 |
| 30.0 | 3.0 | 6 |
| 30.6 | 2.9 | 9 |

dicitrate

| | | |
|---|---|---|
| 10.1 | 8.7 | 40 |
| 12.0 | 7.4 | 100 |
| 12.8 | 6.9 | 39 |
| 14.1 | 6.3 | 28 |
| 15.9 | 5.6 | 24 |
| 16.8 | 5.3 | 12 |
| 17.1 | 5.2 | 32 |
| 17.9 | 5.0 | 11 |
| 18.2 | 4.9 | 17 |
| 19.0 | 4.7 | 58 |
| 19.3 | 4.6 | 45 |
| 19.6 | 4.5 | 52 |
| 20.2 | 4.4 | 13 |
| 20.5 | 4.3 | 66 |
| 21.1 | 4.2 | 21 |
| 22.1 | 4.0 | 8 |
| 22.8 | 3.9 | 41 |
| 23.5 | 3.8 | 16 |
| 24.1 | 3.7 | 11 |
| 25.9 | 3.4 | 9 |
| 26.2 | 3.4 | 13 |
| 27.0 | 3.3 | 15 |
| 27.4 | 3.3 | 11 |
| 28.8 | 3.1 | 8 |
| 30.4 | 2.9 | 8 |

monocitrate Form A

| | | |
|---|---|---|
| 3.4 | 26.2 | 14 |
| 9.4 | 9.4 | 35 |
| 10.2 | 8.7 | 6 |
| 11.1 | 8.0 | 54 |
| 11.8 | 7.5 | 12 |
| 12.1 | 7.3 | 23 |
| 13.5 | 6.6 | 59 |
| 15.9 | 5.6 | 20 |
| 16.2 | 5.5 | 13 |
| 16.6 | 5.3 | 25 |
| 18.0 | 4.9 | 47 |
| 18.8 | 4.7 | 32 |
| 19.5 | 4.5 | 100 |
| 19.8 | 4.5 | 82 |
| 20.3 | 4.4 | 34 |
| 21.5 | 4.1 | 58 |
| 22.3 | 4.0 | 8 |
| 24.3 | 3.7 | 22 |
| 24.6 | 3.6 | 22 |
| 25.8 | 3.5 | 18 |
| 26.5 | 3.4 | 8 |
| 26.6 | 3.3 | 10 |
| 27.9 | 3.2 | 12 |
| 30.3 | 3.0 | 15 |
| 32.3 | 2.8 | 11 |
| 33.4 | 2.7 | 8 |

monocitrate Form B

| | | |
|---|---|---|
| 3.4 | 25.5 | 18 |
| 9.5 | 9.3 | 27 |
| 10.4 | 8.5 | 7 |
| 11.3 | 7.8 | 38 |
| 11.9 | 7.5 | 14 |
| 12.2 | 7.2 | 11 |
| 13.4 | 6.6 | 12 |
| 13.7 | 6.4 | 28 |
| 14.0 | 6.3 | 28 |
| 15.5 | 5.7 | 13 |
| 15.8 | 5.6 | 17 |
| 16.4 | 5.4 | 20 |
| 17.8 | 5.0 | 32 |
| 18.1 | 4.9 | 13 |
| 19.1 | 4.7 | 100 |
| 19.5 | 4.6 | 27 |
| 19.9 | 4.5 | 35 |
| 20.4 | 4.3 | 48 |
| 21.0 | 4.2 | 7 |
| 21.9 | 4.1 | 10 |
| 22.3 | 4.0 | 38 |
| 22.7 | 3.9 | 12 |
| 24.4 | 3.6 | 20 |
| 25.1 | 3.6 | 17 |
| 26.0 | 3.4 | 17 |
| 28.3 | 3.2 | 10 |
| 31.2 | 2.9 | 8 |
| 32.4 | 2.8 | 5 |

dihydrobromide

| | | |
|---|---|---|
| 2.8 | 31.6 | 18 |
| 5.6 | 15.7 | 17 |
| 8.4 | 10.5 | 8 |
| 11.3 | 7.8 | 24 |
| 14.1 | 6.3 | 7 |
| 14.9 | 6.0 | 23 |
| 15.4 | 5.8 | 17 |
| 16.4 | 5.4 | 33 |
| 16.7 | 5.3 | 63 |
| 17.0 | 5.2 | 100 |
| 17.7 | 5.0 | 17 |
| 19.5 | 4.5 | 9 |
| 20.2 | 4.4 | 22 |
| 21.6 | 4.1 | 7 |
| 22.0 | 4.0 | 33 |
| 23.7 | 3.8 | 11 |
| 24.7 | 3.6 | 88 |
| 26.0 | 3.4 | 35 |
| 27.2 | 3.3 | 7 |
| 28.5 | 3.1 | 38 |
| 29.0 | 3.1 | 10 |
| 29.9 | 3.0 | 21 |
| 30.4 | 2.9 | 7 |
| 31.0 | 2.9 | 11 |
| 32.1 | 2.8 | 7 |

oxalate

| | | |
|---|---|---|
| 4.1 | 21.4 | 8 |
| 8.5 | 10.4 | 44 |
| 9.3 | 9.5 | 11 |
| 14.6 | 6.1 | 13 |
| 14.8 | 6.0 | 23 |
| 15.4 | 5.8 | 30 |
| 16.5 | 5.4 | 24 |
| 16.6 | 5.3 | 22 |
| 17.4 | 5.1 | 30 |
| 18.7 | 4.7 | 13 |
| 20.8 | 4.3 | 100 |
| 21.6 | 4.1 | 12 |
| 22.3 | 4.0 | 29 |
| 22.5 | 3.9 | 50 |
| 23.6 | 3.8 | 23 |
| 26.7 | 3.3 | 14 |
| 27.4 | 3.3 | 14 |
| 29.0 | 3.1 | 37 |
| 37.0 | 2.4 | 8 |

sulfate

| | | |
|---|---|---|
| 7.8 | 11.4 | 5 |
| 11.4 | 7.7 | 8 |
| 11.7 | 7.6 | 10 |
| 12.6 | 7.0 | 6 |
| 13.4 | 6.6 | 3 |
| 14.2 | 6.2 | 2 |
| 14.7 | 6.0 | 6 |
| 15.6 | 5.7 | 19 |
| 16.1 | 5.5 | 3 |
| 16.5 | 5.4 | 18 |
| 17.1 | 5.2 | 9 |
| 17.7 | 5.0 | 9 |
| 18.0 | 4.9 | 37 |
| 18.5 | 4.8 | 77 |
| 19.6 | 4.5 | 40 |
| 19.9 | 4.5 | 55 |
| 21.0 | 4.2 | 5 |
| 21.6 | 4.1 | 3 |
| 22.5 | 4.0 | 11 |
| 22.9 | 3.9 | 100 |
| 23.9 | 3.7 | 9 |
| 24.4 | 3.6 | 4 |
| 25.2 | 3.5 | 12 |
| 25.5 | 3.5 | 8 |
| 27.5 | 3.2 | 13 |
| 28.3 | 3.2 | 12 |
| 28.7 | 3.1 | 4 |
| 30.4 | 2.9 | 3 |
| 32.8 | 2.7 | 3 |

TABLE 4
Characteristic peaks measured in IR spectra of the 1-[4-(4-
{3-[(2R)-2-methyl-pyrrolidin-1-yl]-propoxy}-phenoxy)-
piperidin-1-yl]-ethanone crystalline salts and polymorphs thereof [cm⁻¹].

| | | Monocitrate | Monocitrate | | | | HCl | |
|---|---|---|---|---|---|---|---|---|
| Peaks | Dicitrate | Form A | Form B | Oxalate | Sulfate | diHCl | Form B | diHBr |
| 1 | 3523 | 3466 | 2962 | 3431 | 3389 | 3364 | 3429 | 3419 |
| 2 | 3038 | 3011 | 2872 | 2938 | 2955 | 3044 | 2953 | 3043 |
| 3 | 2951 | 2962 | 1732 | 2881 | 2615 | 2959 | 2695 | 2958 |
| 4 | 2521 | 2859 | 1637 | 2513 | 2513 | 2931 | 1621 | 2930 |
| 5 | 1966 | 1731 | 1595 | 1987 | 1590 | 2903 | 1507 | 2901 |
| 6 | 1727 | 1618 | 1508 | 1706 | 1507 | 2874 | 1456 | 2843 |
| 7 | 1687 | 1594 | 1477 | 1693 | 1487 | 2604 | 1392 | 2614 |
| 8 | 1587 | 1507 | 1452 | 1624 | 1453 | 2519 | 1366 | 2520 |
| 9 | 1507 | 1479 | 1398 | 1507 | 1374 | 2184 | 1271 | 1840 |
| 10 | 1489 | 1454 | 1367 | 1470 | 1360 | 1773 | 1218 | 1685 |
| 11 | 1475 | 1394 | 1316 | 1455 | 1269 | 1682 | 1131 | 1616 |
| 12 | 1426 | 1318 | 1288 | 1367 | 1220 | 1616 | 1118 | 1508 |
| 13 | 1394 | 1285 | 1271 | 1220 | 1112 | 1508 | 1038 | 1469 |
| 14 | 1358 | 1270 | 1217 | 1134 | 1044 | 1468 | 998 | 1441 |
| 15 | 1315 | 1217 | 1172 | 1044 | 1010 | 1441 | 970 | 1413 |
| 16 | 1276 | 1175 | 1131 | 1008 | 975 | 1422 | 829 | 1337 |
| 17 | 1216 | 1133 | 1068 | 976 | 924 | 1337 | 781 | 1284 |
| 18 | 1130 | 1043 | 1043 | 948 | 855 | 1285 | 748 | 1232 |
| 19 | 1116 | 1004 | 1000 | 832 | 816 | 1232 | 595 | 1133 |
| 20 | 1062 | 974 | 975 | 805 | 777 | 1162 | 527 | 1117 |
| 21 | 1030 | 937 | 951 | 752 | 722 | 1118 | | 1091 |
| 22 | 1000 | 908 | 908 | 722 | 646 | 1092 | | 1074 |
| 23 | 962 | 847 | 818 | 648 | 624 | 1075 | | 1053 |
| 24 | 948 | 806 | 746 | 593 | 591 | 1053 | | 1033 |
| 25 | 933 | 771 | 664 | 478 | 516 | 1030 | | 1005 |
| 26 | 872 | 746 | 601 | | 448 | 1006 | | 938 |
| 27 | 825 | 665 | 521 | | 436 | 975 | | 949 |
| 28 | 805 | 601 | 489 | | | 939 | | 938 |
| 29 | 782 | 535 | 434 | | | 918 | | 817 |
| 30 | 736 | 488 | | | | 897 | | 755 |
| 31 | 695 | 434 | | | | 818 | | 718 |
| 32 | 661 | | | | | 754 | | 670 |
| 33 | 606 | | | | | 722 | | 584 |
| 34 | 588 | | | | | 671 | | 571 |
| 35 | 497 | | | | | 576 | | 516 |
| 36 | 477 | | | | | 516 | | 495 |
| 37 | 443 | | | | | 496 | | |

TABLE 5
Characteristic peaks measured in Raman spectra of the 1-[4-(4-
{3-[(2R)-2-methyl-pyrrolidin-1-yl]-propoxy}-phenoxy)-
piperidin-1-yl]-ethanone crystalline salts and polymorphs thereof [cm⁻¹].

| | | Monocitrate | Monocitrate | | | | HCl | |
|---|---|---|---|---|---|---|---|---|
| Peaks | Dicitrate | Form A | Form B | Oxalate | Sulfate | diHCl | Form B | diHBr |
| 1 | 3070 | 3065 | 3086 | 3077 | 3076 | 3073 | 3074 | 3072 |
| 2 | 3011 | 3012 | 3011 | 3033 | 3059 | 3048 | 3051 | 3045 |
| 3 | 2971 | 2979 | 2982 | 2984 | 2992 | 3023 | 2981 | 3022 |
| 4 | 2933 | 2961 | 2961 | 2935 | 2935 | 2958 | 2932 | 2983 |
| 5 | 1719 | 2931 | 2930 | 2883 | 2907 | 2935 | 2875 | 2958 |
| 6 | 1611 | 2905 | 2881 | 2757 | 2893 | 2878 | 2767 | 2935 |
| 7 | 1585 | 2888 | 2853 | 1733 | 1613 | 2766 | 1615 | 2902 |
| 8 | 1489 | 2849 | 1718 | 1707 | 1584 | 1662 | 1583 | 2876 |
| 9 | 1457 | 1715 | 1628 | 1611 | 1487 | 1614 | 1469 | 1687 |
| 10 | 1394 | 1618 | 1615 | 1477 | 1453 | 1583 | 1442 | 1613 |
| 11 | 1274 | 1603 | 1586 | 1443 | 1378 | 1468 | 1362 | 1583 |
| 12 | 1256 | 1585 | 1476 | 1364 | 1360 | 1442 | 1311 | 1469 |
| 13 | 1191 | 1480 | 1454 | 1305 | 1304 | 1325 | 1257 | 1442 |
| 14 | 1158 | 1457 | 1365 | 1268 | 1257 | 1312 | 1164 | 1265 |
| 15 | 1132 | 1438 | 1307 | 1246 | 1172 | 1266 | 1133 | 1198 |
| 16 | 1053 | 1370 | 1251 | 1203 | 1152 | 1198 | 1095 | 1164 |
| 17 | 1031 | 1304 | 1169 | 1178 | 1133 | 1163 | 1041 | 1034 |
| 18 | 1000 | 1283 | 1134 | 1157 | 1106 | 1093 | 1005 | 910 |
| 19 | 936 | 1252 | 1043 | 1137 | 1053 | 1068 | 910 | 852 |
| 20 | 910 | 1242 | 999 | 1108 | 1011 | 1034 | 886 | 843 |
| 21 | 854 | 1169 | 930 | 1068 | 961 | 911 | 853 | 719 |
| 22 | 782 | 1134 | 857 | 1044 | 926 | 853 | 837 | 671 |
| 23 | 720 | 1053 | 718 | 1009 | 854 | 809 | 724 | 638 |
| 24 | 660 | 1039 | 665 | 981 | 821 | 722 | 668 | 519 |
| 25 | 640 | 1006 | 647 | 958 | 792 | 706 | 638 | 378 |
| 26 | 607 | 950 | 602 | 928 | 723 | 672 | 518 | 319 |
| 27 | 516 | 935 | 513 | 858 | 706 | 638 | 464 | 261 |
| 28 | 460 | 859 | 380 | 842 | 644 | 517 | 413 | |
| 29 | 371 | 840 | 308 | 807 | 625 | 496 | 378 | |
| 30 | 314 | 804 | 253 | 745 | 581 | 466 | | |
| 31 | 257 | 722 | | 725 | 519 | 418 | | |
| 32 | | 665 | | 708 | 482 | 379 | | |
| 33 | | 642 | | 648 | 469 | 299 | | |
| 34 | | 619 | | 621 | 420 | | | |
| 35 | | 515 | | 514 | 381 | | | |
| 36 | | 470 | | 489 | | | | |
| 37 | | 389 | | 456 | | | | |
| 38 | | 309 | | 428 | | | | |
| 39 | | 265 | | 382 | | | | |
| 40 | | | | 329 | | | | |
| 41 | | | | 298 | | | | |
| 42 | | | | 222 | | | | |

For solid phase analytical studies, the following experimental conditions were used:

### Parameters of FT-IR Spectroscopy Measurements:

| Device | Thermo-Nicolet 6700 |
|---|---|
| Phase | KBr pastille |
| Spectral resolution | 4 cm⁻¹ |
| Detector | DTGS |
| Beamsplitter | XT-KBr |
| Mirror movement speed | 0.6329 |
| Number of scans | 100 |

### Parameters of FT-Raman Spectroscopy Measurements:

| Device | Thermo-Nicolet NXR9650 | |
|---|---|---|
| Measurement range | 3500 to 200 | cm⁻¹ |
| Spectral resolution | 4 | cm⁻¹ |
| Detector | Ge | |
| Beamsplitter | CaF₂ | |
| Mirror movement speed | 0.1581 | |
| Number of scans | 256 | |
| Laser performance | 500 | mW |

### Parameters of X-Ray Powder Diffraction Measurements:

| Device | PANanalytical X'Pert PRO MPD | |
|---|---|---|
| Radiation | CuKα | |
| Accelerating voltage | 40 | kV |
| Anode current | 40 | mA |
| Goniometer | PW3050/60 | |
| Scanning speed | 0.0305°/s | |
| Increment | 0.0131° | |
| Sample holder | PW1818/25 & 40 | |
| | (transmission, sample between foils) | |
| Sample holder spinner | PW3064/60 (reflection/transmission spinner) | |
| Spinning speed of sample | 1 | spin/s |
| holder | | |

| | |
|---|---|
| Detector | PIXcel (PW3018/00) |
| The uncertainty of the 2θ | ±0.2° |
| measurement | |

### Parameters of TG Measurements:

| Device | TA Instruments TGA Q5000 or Discovery TGA 5500 | |
|---|---|---|
| Heating speed | 10° | C./min |
| Sample weight | ~5 to 10 | mg |
| Atmosphere | 60 | mL/min N₂ |

### Parameters of DSC Measurements:

| Device | TA Instruments DSC Q1000 or Discovery DSC 2500 | |
|---|---|---|
| Heating speed | 10° | C./min |
| Sample weight | ~1 to 2 | mg |
| Type of jar | open Al jar | |
| Atmosphere | 50 | mL/min N₂ |

### Parameters of DVS Measurements:

| Device | SMS DVS Advantage 1 | |
|---|---|---|
| dm/dt criteria | 0.002%/min | |
| time limit max./min. | 360 min/10 min | |
| Temperature | 25° | C. |
| Cycle | 0-5-10-20-30-40-50-60-70-80-90-95- | |
| | 90-80-70-60-50-40-30-20-10-5-0% RH | |
| Gas flow | 150 | mL/min N₂ |

| | |
|---|---|
| Solvent | water |

### Pharmaceutical Compositions

The salts of the present invention may be administered in any pharmaceutically acceptable manner, for example, orally, parenterally, buccally, sublingually, nasally, rectally or transdermally, appropriately to the formulation of the pharmaceutical composition. The therapeutically effective dose is between 0.01 and 40 mg/day.
The following formulation examples illustrate the pharmaceutical compositions of the present invention.

However, the present invention is not limited to these compositions.

### A) Solid Oral Dosage Form

Tablet

| | |
|---|---|
| Active ingredient(s) | 0.005-90% |
| Filler | 1-99.9% |
| Binder | 0-20% |
| Desintegrant | 0-20% |
| Lubricant | 0-10% |
| Other specific excipient(s) | 0-50% |

### B) Parenteral Dosage Form

Intravenous Injection

| | |
|---|---|
| Active ingredient(s) | 0.001-50% |
| Solvent | 10-99.9% |
| Co-solvent | 0-99.9% |
| Osmotic agent | 0-50% |
| Buffer | q.s. |

### C) Other Dosage Form

Suppository

| | |
|---|---|
| Active ingredient(s) | 0.0003-50% |
| Suppository base | 1-99.9% |
| Surface-active agents | 0-20% |
| Lubricant | 0-20% |
| Preservative | q.s. |

### EXAMPLES

The invention is illustrated by the following Reference and working Examples without limiting the scope of the present invention.

### REFERENCE EXAMPLES

### Example 1

### 1-[4-(4-{3-[(2R)-2-methyl-pyrrolidin-1-yl]-propoxy}-phenoxy)-piperidin-1-yl]-ethanone

40 g of 1-[4-(4-{3-[(2R)-2-methyl-pyrrolidin-1-yl]-propoxy}-phenoxy)-piperidin-1-yl]-ethanone hydrochloride salt prepared according to Example 11 of WO 2014/136075 was dissolved in 480 mL of dichloromethane at 0 to 5° C., and then 168 mL of 1M aqueous NaOH was added. After stirring for 10 minutes, the aqueous and organic phases were separated and the organic phase was washed twice with 120 mL of deionized water, dried over 25 g of natrium sulfate and filtered. The solution was concentrated in vacuo to an oil. Evaporation residue: 32.8 g of an oil.

### Example 2

### 1-[4-(4-{3-[(2R)-2-methyl-pyrrolidin-1-yl]-propoxy}-phenoxy)-piperidin-1-yl]-ethanone dihydrochloride salt

2.0 g (5.55 mmol) of 1-[4-(4-{3-[(2R)-2-methyl-pyrrolidin-1-yl]-propoxy}-phenoxy)-piperidin-1-yl]-ethanone was dissolved in 20 mL of acetone at room temperature. The mixture was cooled to 0 to 5° C. and 0.8 mL of ≥37% hydrochloric acid solution was added dropwise. After stirring for 30 minutes at 0 to 5° C., the crystals were filtered, covered with 1.5 mL of cold acetone, and dried at room temperature.

White crystalline material. Yield: 1.7 g.

### Example 3

### 1-[4-(4-{3-[(2R)-2-methyl-pyrrolidin-1-yl]-propoxy}-phenoxy)-piperidin-1-yl]-ethanone dihydrochloride salt

0.548 g of 1-[4-(4-{3-[(2R)-2-methyl-pyrrolidin-1-yl]-propoxy}-phenoxy)-piperidin-1-yl]-ethanone was dissolved in 1.1 mL of isopropanol at room temperature. To the solution of the base, 0.391 g of 30% hydrochloric acid isopropanol was added dropwise at room temperature. The precipitated slurry was filtered, and then dried for 2 hours under vacuum under nitrogen at 40° C. Yield: 0.42 g.

### Example 4

### 1-[4-(4-{3-[(2R)-2-methyl-pyrrolidin-1-yl]-propoxy}-phenoxy)-piperidin-1-yl]-ethanone monohydrochloride salt Form A

11.831 mg of 1-[4-(4-{3-[(2R)-2-methyl-pyrrolidin-1-yl]-propoxy}-phenoxy)-piperidin-1-yl]-ethanone dihydrochloride salt was weighed in platinum jar and heat treated in TA Instruments TGA Q50 device until elimination of 1 mol of HCl, according to the following program:
1. Heating up to 90° C. with 10° C./min heating rate
2. Hold at 90° C. for 103.7 minutes
3. Heating up to 95° C. with 10° C./min heating rate
4. Hold at 95° C. for 12.9 minutes
5. Heating up to 100° C. at 10° C./min heating rate
6. Hold at 100° C. for 180 minutes.

### Example 5

### 1-[4-(4-{3-[(2R)-2-methyl-pyrrolidin-1-yl]-propoxy}-phenoxy)-piperidin-1-yl]-ethanone monohydrochloride salt Form A

0.4 mL of aqueous sodium bicarbonate solution (97.5 mg NaHCO₃/1 mL H₂O) was added to 0.2 g of 1-[4-(4-{3-[(2R)-2-methyl-pyrrolidin-1-yl]-propoxy}-phenoxy)-piperidin-1-yl]-ethanone dihydrochloride salt. With the equimolar base used, 1 mol of HCl was liberated during the effervescence of the solution. 1 mL of 1,4-dioxane was added to the solution and an oil was obtained after evaporation. 20 to 30 mg of oil were mixed with 0.5 mL of methyl ethyl ketone, filtered and precipitated with 0.5 mL of diisopropyl ether to give an oil. It was seeded with the 1-[4-(4-{3-[(2R)-2-methyl-pyrrolidin-1-yl]-propoxy}-phenoxy)-piperidin-1-yl]-ethanone monohydrochloride salt obtained by thermal treatment in Example 4. After crystallization, the product was filtered and dried at room temperature. Yield: 27 mg.

### Example 6

### 1-[4-(4-{3-[(2R)-2-methyl-pyrrolidin-1-yl]-propoxy}-phenoxy)-piperidin-1-yl]-ethanone monohydrochloride salt Form A

0.1 g of 1-[4-(4-{3-[(2R)-2-methyl-pyrrolidin-1-yl]-propoxy}-phenoxy)-piperidin-1-yl]-ethanone dihydrochloride salt was dissolved in 0.2 mL of deionized water and 0.2 mL of aqueous sodium bicarbonate solution (97.5 mg of NaHCO₃/1 mL of H₂O) was added. The resulting solution was concentrated at 50° C. and at 70 mbar, then dissolved in 5 mL of methyl ethyl ketone, filtered and washed with 1 mL of methyl ethyl ketone. To the solution 11.5 mL of diisopropyl ether was added and seeded with the product of Example 4, an oily precipitation was observed. The solution was concentrated to dryness, the “residue” was dissolved in 1 mL of dimethylformamide and 15 mL of methyl tert-butyl ether was added and then seeded with the product of Example 5. The next day, the precipitated crystalline product was isolated by filtration. Yield: 25 mg.

### Example 7

### 1-[4-(4-{3-[(2R)-2-methyl-pyrrolidin-1-yl]-propoxy}-phenoxy)-piperidin-1-yl]-ethanone monohydrochloride salt Form B

2.0 g of 1-[4-(4-{3-[(2R)-2-methyl-pyrrolidin-1-yl]-propoxy}-phenoxy)-piperidin-1-yl]-ethanone base was dissolved in 20 mL of acetone at room temperature. The mixture was cooled to 0 to 5° C. and 0.4 mL of ≥37% hydrochloric acid solution was added dropwise. After 30 minutes of stirring at 0 to 5° C. it was concentrated to constant weight in a water bath at 40° C. under vacuum. Then, twice 30 mL of toluene was evaporated. White crystalline material. Yield: 1.5 g.

### Example 8

### 1-[4-(4-{3-[(2R)-2-methyl-pyrrolidin-1-yl]-propoxy}-phenoxy)-piperidin-1-yl]-ethanone monohydrochloride salt Form B

0.548 g of 1-[4-(4-{3-[(2R)-2-methyl-pyrrolidin-1-yl]-propoxy}-phenoxy)-piperidin-1-yl]-ethanone base was dissolved in 0.55 mL of methyl tert-butyl ether at room temperature. Slowly, 0.18 g of 30% hydrochloric acid isopropanol was added dropwise at room temperature to the solution of the base. The initially biphasic mixture became miscible with stirring and then converted to a thick crystalline suspension. The precipitated suspension was filtered and dried for 2 hours under vacuum under nitrogen at 40° C. Yield: 0.33 g.

### WORKING EXAMPLES

### Example 9

### 1-[4-(4-{3-[(2R)-2-methyl-pyrrolidin-1-yl]-propoxy}-phenoxy)-piperidin-1-yl]-ethanone dihydrobromide salt

0.53 g of 1-[4-(4-{3-[(2R)-2-methyl-pyrrolidin-1-yl]-propoxy}-phenoxy)-piperidin-1-yl]-ethanone base was dissolved in 5 mL of ethyl acetate at room temperature, followed by the addition of a solution of acetic acid saturated with 0.8 mL of hydrobromic acid the salt was formed. After filtration it was washed twice with 1 mL of acetic acid saturated with hydrobromic acid. The dried product weighed 0.65 g.

### Example 10

### 1-[4-(4-{3-[(2R)-2-methyl-pyrrolidin-1-yl]-propoxy}-phenoxy)-piperidin-1-yl]-ethanone sulfate salt

0.1 g of 1-[4-(4-{3-[(2R)-2-methyl-pyrrolidin-1-yl]-propoxy}-phenoxy)-piperidin-1-yl]-ethanone base oil was dissolved in 9 mL of acetone at room temperature and then 0.125 mL of 20.4% H₂SO₄ solution was slowly added dropwise. The resulting solution first became opalescent, then a crystalline suspension was obtained which was stirred at room temperature for 2 hours. The product was filtered and washed twice with 0.5 mL of acetone. It was dried under vacuum at 40° C. for 2 hours under nitrogen. Yield: 0.08 g.

### Example 11

### 1-[4-(4-{3-[(2R)-2-methyl-pyrrolidin-1-yl]-propoxy}-phenoxy)-piperidin-1-yl]-ethanone sulfate salt

0.99 g of 1-[4-(4-{3-[(2R)-2-methyl-pyrrolidin-1-yl]-propoxy}-phenoxy)-piperidin-1-yl]-ethanone base oil was dissolved in 20 mL of acetone, then 1.3 mL of 18.4% H₂SO₄ solution was added. The mixture was seeded with the product of Example 10 (with the addition of 0.05 mL of water). The product was precipitated with 20 mL of acetone, stirred for half an hour, filtered, washed and dried at 40° C. under nitrogen. Yield: 0.814 g. Melting point of the product (based on DSC peak): 79.5° C.

### Example 12

### 1-[4-(4-{3-[(2R)-2-methyl-pyrrolidin-1-yl]-propoxy}-phenoxy)-piperidin-1-yl]-ethanone sulfate salt

1.008 g of 1-[4-(4-{3-[(2R)-2-methyl-pyrrolidin-1-yl]-propoxy}-phenoxy)-piperidin-1-yl]-ethanone base oil was mixed with 0.935 mL of 3M H₂SO₄ and stirred for 15 minutes. 20 mL of acetone was added and seeded with the product of Example 11 and then stirred at room temperature overnight. Filtered, dried under nitrogen at 40° C. to constant weight. Yield: 0.895 g. Melting point of the product (based on DSC peak): 79.3° C.

### Example 13

### 1-[4-(4-{3-[(2R)-2-methyl-pyrrolidin-1-yl]-propoxy}-phenoxy)-piperidin-1-yl]-ethanone oxalate salt

0.1 g of 1-[4-(4-{3-[(2R)-2-methyl-pyrrolidin-1-yl]-propoxy}-phenoxy)-piperidin-1-yl]-ethanone base oil was dissolved in 0.1 mL of acetone at room temperature and a solution of 0.055 g of oxalic acid in 0.5 mL of acetone was added. The product was precipitated with 0.3 mL of ethyl acetate. Filtered and then dried under nitrogen to constant weight. Yield: 128 mg. Melting point of the product (based on DSC peak): 54.2° C.

### Example 14

### 1-[4-(4-{3-[(2R)-2-methyl-pyrrolidin-1-yl]-propoxy}-phenoxy)-piperidin-1-yl]-ethanone oxalate salt

0.5 g of 1-[4-(4-{3-[(2R)-2-methyl-pyrrolidin-1-yl]-propoxy}-phenoxy)-piperidin-1-yl]-ethanone base oil was dissolved 0.5 mL of acetone, and then a solution of 0.275 g of oxalic acid in 1.5 mL of acetone and 0.05 mL of water was added. The precipitated material was filtered and then stirred in a mixture of 0.05 mL of water and 2.75 mL of acetone in the presence of 0.1755 g of oxalic acid. The product obtained was filtered and dried. Yield: 392 mg.

### Example 15

### 1-[4-(4-{3-[(2R)-2-methyl-pyrrolidin-1-yl]-propoxy}-phenoxy)-piperidin-1-yl]-ethanone monocitrate salt Form A

To 0.13 g of 1-[4-(4-{3-[(2R)-2-methyl-pyrrolidin-1-yl]-propoxy}-phenoxy)-piperidin-1-yl]-ethanone base oil 0.081 g of citric acid monohydrate was added at room temperature with stirring. 0.5 mL of acetone was added and stirred overnight. After the addition of further 1 mL of acetone on the following day, the mixture was stirred for an additional 30 minutes, then filtered and washed with 0.5 mL of acetone. The resulting sample was dried under vacuum under nitrogen at 25° C. Yield: 0.163 g.

### Example 16

### 1-[4-(4-{3-[(2R)-2-methyl-pyrrolidin-1-yl]-propoxy}-phenoxy)-piperidin-1-yl]-ethanone monocitrate salt Form A

To 0.513 g of 1-[4-(4-{3-[(2R)-2-methyl-pyrrolidin-1-yl]-propoxy}-phenoxy)-piperidin-1-yl]-ethanone base oil a solution of 0.315 g citric acid monohydrate in 5 mL of acetone was added at room temperature with stirring. The solution was seeded with the product of Example 15. After stirring for two hours, another 2 mL of acetone was added and stirred for a weekend. The mixture was filtered and washed with 5 mL of acetone. The resulting crystalline material was dried under vacuum under nitrogen at 25° C. Yield 0.63 g. Karl-Fischer water content: 3.1%.

### Example 17

### 1-[4-(4-{3-[(2R)-2-methyl-pyrrolidin-1-yl]-propoxy}-phenoxy)-piperidin-1-yl]-ethanone monocitrate salt Form A

1.020 g of 1-[4-(4-{3-[(2R)-2-methyl-pyrrolidin-1-yl]-propoxy}-phenoxy)-piperidin-1-yl]-ethanonebase oil was weighted to a 100 mL reactor and stirred with 15 mL of acetone at room temperature. To this solution 15 mL of a solution of citric acid monohydrate (0.872 g of citric acid monohydrate dissolved in 20 mL of acetone) was added at room temperature. In the meantime, it was seeded with a suspension of the monocitrate salt prepared in Example 16 (0.0767 g suspended in 0.5 mL of acetone). The resulting suspension was stirred at room temperature for 1 hour, then the precipitated salt was filtered and washed with 10 mL of acetone. The resulting crystalline material was dried at 25° C. under nitrogen.

Yield: 1.385 g. Melting point of the product (DSC onset): 114.3° C. Karl-Fischer water content: 3.5%.

### Example 18

### 1-[4-(4-{3-[(2R)-2-methyl-pyrrolidin-1-yl]-propoxy}-phenoxy)-piperidin-1-yl]-ethanone monocitrate salt Form B

The monocitrate salt Form A of Example 17 was dried at 70 to 90° C. under nitrogen to constant weight.

### Example 19

### 1-[4-(4-{3-[(2R)-2-methyl-pyrrolidin-1-yl]-propoxy}-phenoxy)-piperidin-1-yl]-ethanone dicitrate salt

0.5 g of 1-[4-(4-{3-[(2R)-2-methyl-pyrrolidin-1-yl]-propoxy}-phenoxy)-piperidin-1-yl]-ethanone base oil was dissolved in 1 mL of acetone, to which a solution of 0.962 g of citric acid monohydrate in 4 mL of acetone was added. After stirring for 1 h 15 min at reflux temperature, it was cooled to room temperature, then filtered and washed with 10 mL of acetone. The resulting sample was dried overnight at 25° C. under vacuum under nitrogen. Yield: 0.832 mg.

### Example 20

### 1-[4-(4-{3-[(2R)-2-methyl-pyrrolidin-1-yl]-propoxy}-phenoxy)-piperidin-1-yl]-ethanone dicitrate salt

1.928 g of citric acid monohydrate was added to a 100 mL reactor and dissolved in 15 mL of acetone at room temperature. To this solution an acetone suspension (0.1039 g/0.5 mL) of the dicitrate salt prepared in Example 19 was added. To this solution a solution of 15 mL of the base in acetone (1.695 g of 1-[4-(4-{3-[(2R)-2-methyl-pyrrolidin-1-yl]-propoxy}-phenoxy)-piperidin-1-yl]-ethanone base oil dissolved in 17.5 mL of acetone) was added. The solution was stirred at room temperature for 1 hour, then filtered off and washed with 10 mL of acetone. The resulting sample was dried for 1 day at 25° C. under nitrogen. Yield: 2.81 g. Melting point of the product (DSC onset): 133.1° C. Karl-Fischer water content: 0.4%.

### Example 21

### 1-[4-(4-{3-[(2R)-2-methyl-pyrrolidin-1-yl]-propoxy}-phenoxy)-piperidin-1-yl]-ethanone dicitrate salt

17.6 kg of dichloromethane was introduced into the reactor and then inertized with nitrogen and the temperature was set to 0 to 5° C. 1.1 kg of 1-[4-(4-{3-[(2R)-2-methyl-pyrrolidin-1-yl]-propoxy}-phenoxy)-piperidin-1-yl]-ethanone dihydrochloride was added, and then a mixture of 5.7 kg of purified water and 0.19 kg of NaOH was added while maintaining the temperature at 0 to 5° C. After a reaction time of 15 to 20 minutes, the organic phase was conducted to another reactor, which was also inertized with nitrogen. 2200 mL of dichloromethane was added to the organic phase and, after stirring for 30 to 40 minutes, the organic phase was separated again. The following step was repeated twice: 3300 mL of purified water was added to the organic phase and after 30 to 40 minutes of stirring, the organic phase was separated again. A solution of 0.66 kg of NaCl in 2.6 L of purified water was added to the separated organic phase and, after stirring for 30 to 40 minutes, the organic phase was separated again. The organic phase was concentrated under 0.5 bar vacuum at max. 35° C. to the stirring limit (to 3 to 4 liters). Repeated three times, 8.8 kg acetone was added and the liberated base was concentrated under 0.7 bar vacuum at max. 45° C. to the stirring limit (to 3 to 4 liters). 1.1 kg of citric acid monohydrate was dissolved in 7.0 kg of acetone while maintaining the temperature of the solution at 20 to 25° C. To the resulting citric acid solution 5.0 g of 1-[4-(4-{3-[(2R)-2-methyl-pyrrolidin-1-yl]-propoxy}-phenoxy)-piperidin-1-yl]-ethanone dicitrate seed crystals were added. To the resulting solution the solution of the concentrated 1-[4-(4-{3-[(2R)-2-methyl-pyrrolidin-1-yl]-propoxy}-phenoxy)-piperidin-1-yl]-ethanone base in acetone was added over 110 to 130 minutes, keeping the temperature between 20 to 25° C. After addition, the mixture was heated to 55 to 60° C. and stirred at this temperature for 10 to 12 minutes and then cooled to 20 to 25° C. for an additional 10 hours. At the end of the stirring time, the material was centrifuged and dried. Yield: 1.398 kg. Melting point of the product (DSC onset): 132.7° C.

### Example 22

### 1-[4-(4-{3-[(2R)-2-methyl-pyrrolidin-1-yl]-propoxy}-phenoxy)-piperidin-1-yl]-ethanone dicitrate salt

70 g of 1-[4-(4-{3-[(2R)-2-methyl-pyrrolidin-1-yl]-propoxy}-phenoxy)-piperidin-1-yl]-ethanone dihydrochloride salt was weighted and dissolved in 840 mL of dichloromethane at 0 to 5° C., followed by addition of a solution of 11.9 g of NaOH in 350 mL of deionized water. After stirring for 15 minutes, the mixture was separated and the aqueous phase was transferred to 140 mL of dichloromethane. The combined organic phase was extracted twice with 210 mL of deionized water and then with 210 mL of saturated brine. The solution was concentrated to an oil in vacuo (at max. 35° C.). The mixture was diluted three times with 700 mL of acetone and evaporated. The evaporation residue was complemented with acetone to 560 mL. The solution of 1-[4-(4-{3-[(2R)-2-methyl-pyrrolidin-1-yl]-propoxy}-phenoxy)-piperidin-1-yl]-ethanone in acetone was added to a solution of 64 g citric acid anhydrate in 560 mL of acetone and 6 mL of water (20 to 25° C.) over two hours, keeping at 20 to 25° C. After stirring for 15 minutes at reflux, the suspension was cooled back to room temperature and after further stirring for 2 hours, the precipitate was filtered off, washed twice with 60 mL of acetone and dried at 50° C. under vacuum. Yield: 101.6 g. Melting point of the product (DSC onset): 132.6° C.

1. 1-[4-(4-{3-[(2R)-2-methyl-pyrrolidin-1-yl]-propoxy}-phenoxy)-piperidin-1-yl]-ethanone salts characterized in that based on the dynamic vapor sorption (DVS) analysis at 25° C.
a.) showing deliquescence only above 70% relative humidity, i.e. the critical relative humidity (CRH) value, determined in the range of 10 to 90% RH of the sorption curve, indicating of the onset of deliquescence, is at least 70%; or
b.) not showing deliquescence, i.e. characterized by a value of 0.5>Δm/ΔRH in the range of 10 to 90% RH of the sorption curve, wherein
Δm=m2−m1, the difference of the quasi-equilibrium relative mass changes m2 and m1 corresponding to the RH2 and RH1 relative humidity values and wherein
ΔRH=RH2−RH1=10.

2. The salts according to claim 1, wherein the salts are selected from the group consisting of salts formed with hydrogen bromide, sulfuric acid, oxalic acid and citric acid.

3. The salts according to claim 1, wherein the salt is monocitrate salt.

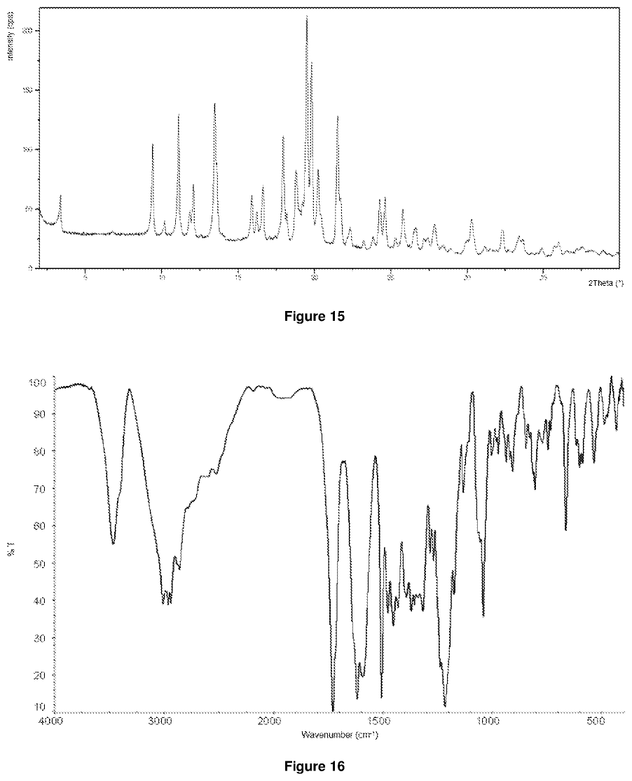

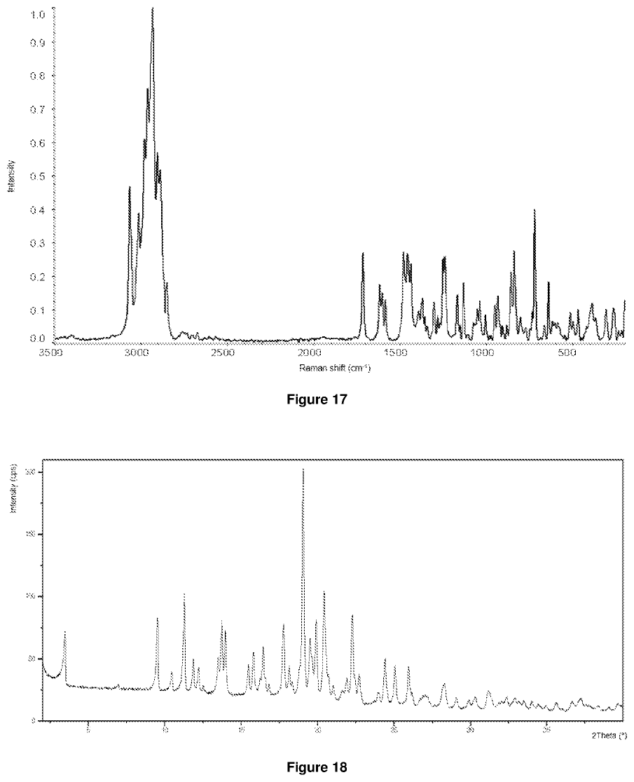

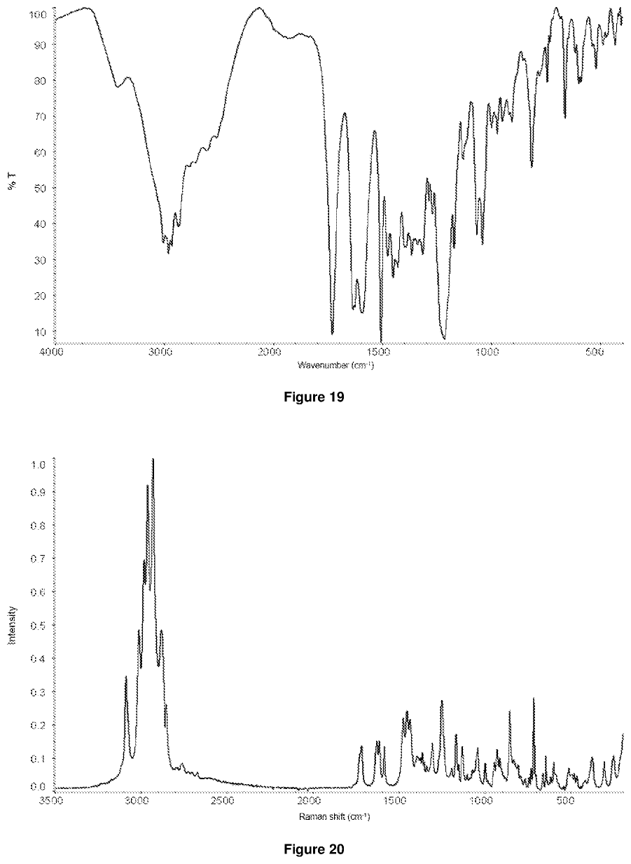

4. The crystalline Form A of 1-[4-(4-{3-[(2R)-2-methyl-pyrrolidin-1-yl]-propoxy}-phenoxy)-piperidin-1-yl]-ethanone monocitrate salt according to claim 3 characterized in that
characteristic reflections in the X-ray powder diffractogram thereof are present in scattering angle value ranges of 9.4; 11.1; 13.5; 18.0; 19.5; 19.8; 21.5±0.2° 2θ,
its X-ray powder diffractogram corresponds to the X-ray powder diffractogram shown in FIG. 15,
absorption bands in the infrared spectra thereof are present in value ranges of 3466; 3011; 2962; 1731; 1618; 1594; 1507; 1217; 1043; 665 cm⁻¹±4 cm⁻¹, or
absorption bands in the Raman spectra thereof are present in value ranges of 3065; 2961; 2931; 1715; 1618; 1480; 1242; 1134; 859; 840; 722 cm⁻¹±4 cm⁻¹.

5-7. (canceled)

8. The crystalline Form B of 1-[4-(4-{3-[(2R)-2-methyl-pyrrolidin-1-yl]-propoxy}-phenoxy)-piperidin-1-yl]-ethanone monocitrate salt according to claim 3 characterized in that
characteristic reflections in the X-ray powder diffractogram thereof are present in scattering angle value ranges of 9.5; 11.3; 13.4; 13.7; 14.0; 17.8; 19.1; 20.4; 22.3°±0.2° 2θ,
its X-ray powder diffractogram corresponds to the X-ray powder diffractogram shown in FIG. 18,
absorption bands in the infrared spectra thereof are present in value ranges of 2962; 2872; 1732; 1637; 1595; 1508; 1217; 1068; 1043; 818 cm⁻¹±4 cm⁻¹, or
absorption bands in the Raman spectra thereof are present in value ranges of 3086; 2930; 2881; 1718; 1628; 1476; 1251; 857; 718 cm⁻¹±4 cm⁻¹.

9-11. (canceled)

12. The salts according to claim 1, wherein the salt is dicitrate salt.

13. The crystalline form of 1-[4-(4-{3-[(2R)-2-methyl-pyrrolidin-1-yl]-propoxy}-phenoxy)-piperidin-1-yl]-ethanone dicitrate salt according to claim 12 characterized in that
characteristic reflections in the X-ray powder diffractogram thereof are present in scattering angle value ranges of 10.2; 12.0; 12.8; 14.1; 19.0; 19.3; 20.5; 22.8°±0.2° 2θ,
its X-ray powder diffractogram corresponds to the X-ray powder diffractogram shown in FIG. 22,
absorption bands in the infrared spectra thereof are present in value ranges of 3523; 3038; 2521; 1727; 1687; 1587; 1507; 1216; 782; 661 cm⁻¹±4 cm⁻¹, or
absorption bands in the Raman spectra thereof are present in value ranges of 3070; 2971; 2933; 1611; 1489; 936; 854; 782; 720; 660 cm⁻¹±4 cm⁻¹.

14-16. (canceled)

17. The salts according to claim 1, wherein the salt is dihydrobromide salt.

18. The crystalline form of 1-[4-(4-{3-[(2R)-2-methyl-pyrrolidin-1-yl]-propoxy}-phenoxy)-piperidin-1-yl]-ethanone dihydrobromide salt according to claim 17 characterized in that
characteristic reflections in the X-ray powder diffractogram thereof are present in scattering angle value ranges of 5.6; 11.3; 16.4; 16.7; 17.0; 24.7; 28.5°±0.2° 2θ,
its X-ray powder diffractogram corresponds to the X-ray powder diffractogram shown in FIG. 26,
absorption bands in the infrared spectra thereof are present in value ranges of 3419; 2930; 2614; 1685; 1616; 1508; 1232; 817 cm⁻¹±4 cm⁻¹, or
absorption bands in the Raman spectra thereof are present in value ranges of 3072; 3022; 3045; 2935; 1613; 1265; 1164; 852 cm⁻¹±4 cm¹.

19-21. (canceled)

22. The salts according to claim 1, wherein the salt is sulfate salt.

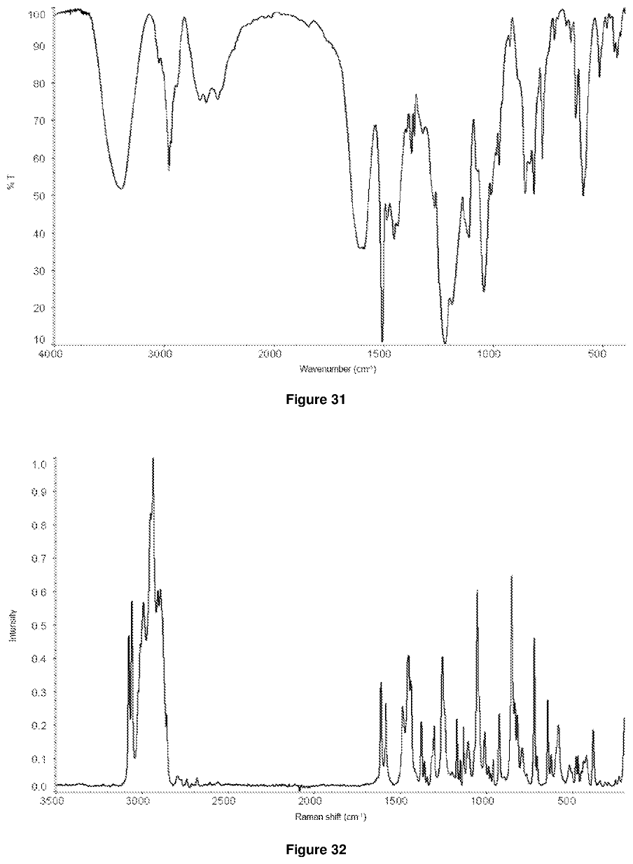

23. The crystalline form of 1-[4-(4-{3-[(2R)-2-methyl-pyrrolidin-1-yl]-propoxy}-phenoxy)-piperidin-1-yl]-ethanone sulfate salt according to claim 22 characterized in that
characteristic reflections in the X-ray powder diffractogram thereof are present in scattering angle value ranges of 7.8; 11.7; 15.6; 18.0; 18.5; 19.6; 19.9; 22.9°±0.2° 2θ,
its X-ray powder diffractogram corresponds to the X-ray powder diffractogram shown in FIG. 30,
absorption bands in the infrared spectra thereof are present in value ranges of 3389; 2955; 2615; 2513; 1590; 1507; 1220; 1044; 855; 591 cm⁻¹±4 cm⁻¹, or
absorption bands in the Raman spectra thereof are present in value ranges of 3076; 3059; 2935; 1613; 1584; 1453; 1257; 1053; 854; 723 cm⁻¹±4 cm⁻¹.

24-26. (canceled)

27. The salts according to claim 1, wherein the salt is oxalate salt.

28. The crystalline form of 1-[4-(4-{3-[(2R)-2-methyl-pyrrolidin-1-yl]-propoxy}-phenoxy)-piperidin-1-yl]-ethanone oxalate salt according to claim 27 characterized in that
characteristic reflections in the X-ray powder diffractogram thereof are present in scattering angle value ranges of 8.5; 14.8; 15.4; 16.5; 17.4; 20.8; 22.5°±0.2° 2θ,
its X-ray powder diffractogram corresponds to the X-ray powder diffractogram shown in FIG. 34,
absorption bands in the infrared spectra thereof are present in value ranges of 3431; 2513; 1987; 1706; 1693; 1507; 1220; 832; 722 cm⁻¹±4 cm⁻¹, or
absorption bands in the Raman spectra thereof are present in value ranges of 3077; 2935; 2883; 1611; 1477; 1443; 858; 842; 725 cm⁻¹±4 cm⁻¹.

29-31. (canceled)

32. Process for the preparation of a compound according to claim 1 characterized in that the 1-[4-(4-{3-[(2R)-2-methyl-pyrrolidin-1-yl]-propoxy}-phenoxy)-piperidin-1-yl]-ethanone base is dissolved in a suitable solvent or mixture of solvents, followed by the addition of the acid or a salt thereof—formed by a base weaker than 1-[4-(4-{3-[(2R)-2-methyl-pyrrolidin-1-yl]-propoxy}-phenoxy)-piperidin-1-yl]-ethanone—or a solution thereof, to the mixture, then the concentration of the reaction mixture is optionally increased, or without it, at room temperature or after cooling, the precipitated product is isolated by filtration.

33. Process for the preparation of a compound according to claim 1 characterized in that the 1-[4-(4-{3-[(2R)-2-methyl-pyrrolidin-1-yl]-propoxy}-phenoxy)-piperidin-1-yl]-ethanone base is dissolved in a suitable solvent or mixture of solvents, followed by the addition of the acid or a salt thereof—formed by a base weaker than 1-[4-(4-{3-[(2R)-2-methyl-pyrrolidin-1-yl]-propoxy}-phenoxy)-piperidin-1-yl]-ethanone—or a solution thereof, to the mixture, then crystallized using a suitable antisolvent added to the solution thus obtained, at room temperature or after cooling, to isolate the precipitated product by filtration.

34. The process according to claim 32 characterized in that 1-[4-(4-{3-[(2R)-2-methyl-pyrrolidin-1-yl]-propoxy}-phenoxy)-piperidin-1-yl]-ethanone monocitrate or dicitrate salt is prepared according to the following process:
(i) dissolving the 1-[4-(4-{3-[(2R)-2-methyl-pyrrolidin-1-yl]-propoxy}-phenoxy)-piperidin-1-yl]-ethanone base in acetone; and
(ii) crystallizing the citrate salt by adding a solution of citric acid in acetone to the solution of the base in acetone.

35. The process according to claim 32 characterized in that 1-[4-(4-{3-[(2R)-2-methyl-pyrrolidin-1-yl]-propoxy}-phenoxy)-piperidin-1-yl]-ethanone monocitrate or dicitrate salt is prepared according to the following process:
(i) dissolving the 1-[4-(4-{3-[(2R)-2-methyl-pyrrolidin-1-yl]-propoxy}-phenoxy)-piperidin-1-yl]-ethanone base in acetone; and
(ii) crystallizing the citrate salt by adding the solution of the base in acetone to a solution of citric acid in acetone.

36. The process according to claim 32 characterized in that 1-[4-(4-{3-[(2R)-2-methyl-pyrrolidin-1-yl]-propoxy}-phenoxy)-piperidin-1-yl]-ethanone dicitrate salt is prepared according to the following process:
(i) starting from the 1-[4-(4-{3-[(2R)-2-methyl-pyrrolidin-1-yl]-propoxy}-phenoxy)-piperidin-1-yl]-ethanone dihydrochloride salt, the base is released and the solution is evaporated after acetone solvent exchange; and
(ii) crystallizing the dicitrate salt by adding the solution of the base in acetone to a solution of citric acid in acetone.

37-38. (canceled)

39. Pharmaceutical composition comprising a therapeutically effective amount of the 1-[4-(4-{3-[(2R)-2-methyl-pyrrolidin-1-yl]-propoxy}-phenoxy)-piperidin-1-yl]-ethanone salts of claim 1 together with one or more pharmaceutically acceptable excipients.

40. A method for the treatment and/or prevention of conditions requiring the modulation of histamine H₃ receptors, such as autism spectrum disorder, said method comprising administering the 1-[4-(4-{3-[(2R)-2-methyl-pyrrolidin-1-yl]-propoxy}-phenoxy)-piperidin-1-yl]-ethanone salt of claim 1, or a pharmaceutical composition comprising a therapeutically effective amount of the salt, to a patient in need thereof.

41. (canceled)
